SEQUENCE LISTING (12 sequences)

SEQ ID NO: 1 - DNA, 21 bp, synthetic construct
  source 1..21: mol_type = other DNA
agctccaaataaccaagatac

SEQ ID NO: 2 - DNA, 22 bp, synthetic construct
  source 1..22: mol_type = other DNA
ctctgctctacactctatctcc

SEQ ID NO: 3 - DNA, 20 bp, synthetic construct
  source 1..20: mol_type = other DNA
gaatgcactctgtagtccag

SEQ ID NO: 4 - DNA, 22 bp, synthetic construct
  source 1..22: mol_type = other DNA
ataaatgaaagtcagctctcag

SEQ ID NO: 5 - DNA, 20 bp, synthetic construct
  source 1..20: mol_type = other DNA
ggaatgcccatgaggtattg

SEQ ID NO: 6 - DNA, 19 bp, synthetic construct
  source 1..19: mol_type = other DNA
agcttgccacattgcattg

SEQ ID NO: 7 - DNA, 21 bp, synthetic construct
  source 1..21: mol_type = other DNA
acatagggaagcaggctaccc

SEQ ID NO: 8 - DNA, 21 bp, synthetic construct
  source 1..21: mol_type = other DNA
ggcaagactctgctggcaagg

SEQ ID NO: 9 - DNA, 22 bp, synthetic construct
  source 1..22: mol_type = other DNA
aagcacagaggcgctgaggcag

SEQ ID NO: 10 - DNA, 21 bp, synthetic construct
  source 1..21: mol_type = other DNA
gactaatttcctaagttcggc

SEQ ID NO: 11 - RNA, 27 nt, synthetic construct
  source 1..27: mol_type = other RNA
cacacttgtgtgcttcacacttgtgtg

SEQ ID NO: 12 - RNA, 27 nt, synthetic construct
  source 1..27: mol_type = other RNA
ccttcttgaaggcttccttcttgaagg

(19) United States
(12) Patent Application Publication
(10) Pub. No.: US 2026/0000643 A1
(43) Pub. Date: Jan. 1, 2026

(54) Combination Of Curaxins And Immune Checkpoint Inhibitors For Treating Cancer

(71) Applicant: Institute For Cancer Research d/b/a The Research Institute of Fox Chase Cancer Center, Philadelphia, PA (US)

(72) Inventors: Siddharth Balachandran, Philadelphia, PA (US); Ting Zhang, Philadelphia, PA (US); Chaoran Yin, Philadelphia, PA (US)

(21) Appl. No.: 18/862,234

(22) PCT Filed: May 8, 2023
(86) PCT No.: PCT/US2023/066717

§ 371 Date: Nov. 1, 2024

Related U.S. Application Data
(60) Provisional application No. 63/339,860, filed on May 9, 2022.

(51) Int. Cl. A61K 31/404; A61K 45/06; A61P 35/00; C07K 16/28

(52) U.S. Cl. CPC A61K 31/404; A61K 45/06; A61P 35/00; C07K 16/2818

(57) ABSTRACT

The present disclosure is directed, in part, to curaxin compounds, or pharmaceutically acceptable salts thereof, combinations of curaxin compounds and immune checkpoint inhibitors, or pharmaceutically acceptable salts thereof, compositions comprising the same, and methods for preventing or treating cancer by administering the same.

### REFERENCE TO SEQUENCE LISTING

This application includes a Sequence Listing filed electronically as an XML file named 853003345SEQ, created on Apr. 28, 2023, with a size of 11,502 bytes. The Sequence Listing is incorporated herein by reference.

### FIELD

The present disclosure is directed, in part, to curaxin compounds, or pharmaceutically acceptable salts thereof. More particularly, the disclosure is directed to combinations of curaxin compounds, or pharmaceutically acceptable salts thereof, and immune checkpoint inhibitors, and compositions comprising the same, and methods for treating cancer by administering the same.

### BACKGROUND

Although immune checkpoint blockade (ICB)-based therapies, such as antibodies to programmed Death (PD)-1, have revolutionized cancer treatment, most (70-90%) patients will either not respond to these therapies, or will eventually develop resistance to them (Topalian et al., N Engl J Med, 2012, 366, 2443-2454; Hodi et al., N Engl J Med, 2010, 363, 711-723; Lu et al., J Oncol Pharm Pract, 2015, 21, 451-467; and Yun et al., Cancer Med, 2016, 5, 1481-1491). One attractive way of inflaming unresponsive (so-called ‘cold’) tumors and overcoming ICB resistance is by activating antiviral innate immune signaling pathways within the tumor microenvironment (TME) (Patel et al., Immunity, 2018, 48, 417-433; and Corrales et al., Cell Res, 2017, 27, 96-108). Reawakening endogenous retroviral elements (EREs) has emerged as a powerful means of stimulating innate immune signaling and kindling ICB responsiveness in cold tumors (Loo et al., Trends Cell Biol, 2019, 29, 31-43). EREs comprise over 50% of mammalian genomes, and their dysregulation results in the production of double-stranded RNA (dsRNA) transcripts, which engage cellular dsRNA sensors and trigger type I interferon (IFN) production, initiating potent antitumor responses (Liu et al., Nat Med, 2019, 25, 95-102; Ishizuka et al., Nature, 2019, 565, 43-48; Mehdipour et al., Nature, 2020, 588, 169-173; Gannon et al. Nat Commun, 2018, 9, 5450; Cuellar et al., J Cell Biol, 2017, 216, 3535-3549; Sheng et al., Cell, 2018, 174, 549-563 e519; Chiappinelli et al., Cell, 2016, 164, 1073; Roulois et al., Cell, 2015, 162, 961-973; Goel et al., Nature, 2017, 548, 471-475; Canadas et al., Nat Med, 2018, 24, 1143-1150; Zhang et al., Cell, 2018, 175, 1244-1258 e1226; and Bowling et al., Cell, 2021).

The enzyme Adenosine Deaminase RNA Specific 1 (ADAR1) is a major repressor of immune responses activated by ERE-derived dsRNAs (Heraud-Farlow et al., Curr Opin Hematol, 2019, 26, 241-248; and Eisenberg et al., Nat Rev Genet, 2018, 19, 473-490). ADAR1 binds dsRNAs and reduces their capacity to activate dsRNA sensors by introducing A-I edits in their sequences (Heraud-Farlow et al., Curr Opin Hematol, 2019, 26, 241-248; Samuel et al., J Biol Chem, 2019, 294, 1710-1720; and George et al., J Biol Chem, 2016, 291, 6158-6168). DsRNAs typically adopt the right-handed (A-RNA) conformation and are recognized by the dsRNA binding domains (dsRBDs) of ADAR1, preventing amplification of immune responses by the host A-RNA sensors Melanoma Differentiation Antigen-5 (MDA-5) and Protein Kinase dsRNA-Dependent (PKR) (Liu et al., Nat Med, 2019, 25, 95-102; Ishizuka et al., Nature, 2019, 565, 43-48; Mehdipour et al., Nature, 2020, 588, 169-173; Gannon et al. Nat Commun, 2018, 9, 5450; George et al., J Biol Chem, 2016, 291, 6158-6168; and Chung et al., Cell, 2018, 172, 811-824 e814). When ADAR1 expression is ablated in tumors, MDA-5 and PKR are activated, resulting in type I IFN cytokine production, loss of tumor cell fitness, and remarkably enhanced ICB responsiveness (Liu et al., Nat Med, 2019, 25, 95-102; Ishizuka et al., Nature, 2019, 565, 43-48; Mehdipour et al., Nature, 2020, 588, 169-173; Gannon et al. Nat Commun, 2018, 9, 5450; and Heraud-Farlow et al., Curr Opin Hematol, 2019, 26, 241-248).

### SUMMARY

The present disclosure provides compounds of Formula I:

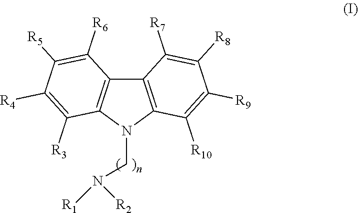

or a pharmaceutically acceptable salt thereof, wherein: each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl; each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and n is 1, 2, or 3.

The present disclosure also provides compounds of Formula II:

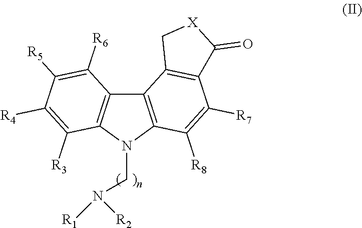

or a pharmaceutically acceptable salt thereof, wherein: each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl; each of R₃, R₄, R₅, R₆, R₇, and R₈ is, independently, selected from the group consisting of hydrogen, halogen, hydroxyl, cyano, nitro, substituted or unsubstituted alkyl, —C(═O)alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; X is substituted or unsubstituted carbon, substituted or unsubstituted nitrogen, or oxygen; and n is 1, 2, or 3.

The present disclosure also provides compounds of Formula III:

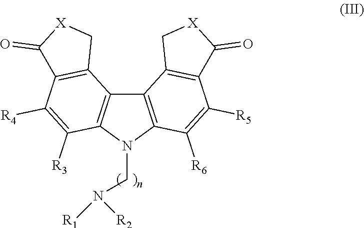

or a pharmaceutically acceptable salt thereof, wherein: each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl; each of R₃, R₄, R₅, and R₆ is, independently, selected from the group consisting of hydrogen, halogen, hydroxyl, cyano, nitro, substituted or unsubstituted alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; X is substituted or unsubstituted carbon, substituted or unsubstituted nitrogen, or oxygen; and n is 1, 2, or 3.

The present disclosure also provides pharmaceutical compositions comprising any one or more of the compounds having Formula I, Formula II, and Formula III, or a pharmaceutically acceptable salt thereof, and an immune checkpoint inhibitor.

The present disclosure also provides methods of treating cancer in a subject comprising administering to the subject in need thereof any one or more of the compounds having Formula I, Formula II, and Formula III, or a pharmaceutically acceptable salt thereof, and an immune checkpoint inhibitor.

The present disclosure also provides uses of any one or more of the compounds having Formula I, Formula II, and Formula III, or a pharmaceutically acceptable salt thereof, or a composition comprising any one or more of the compounds having Formula I, Formula II, and Formula III, or a pharmaceutically acceptable salt thereof, and an immune checkpoint inhibitor, in the preparation of a medicament for treating a subject having cancer.

The present disclosure also provides uses of any one or more of the compounds having Formula I, Formula II, and Formula III, or a pharmaceutically acceptable salt thereof, or a composition comprising any one or more of the compounds having Formula I, Formula IL, and Formula III, or a pharmaceutically acceptable salt thereof, and an immune checkpoint inhibitor for treating a subject having cancer.

### BRIEF DESCRIPTION OF THE DRAWINGS

FIG. 1 (Panel a) depicts ADAR1 protein levels in MEFs following transfection of recombinant CRISPR/Cas9 proteins, together with sgRNAs targeting luciferase (ADAR WT) or murine Adar1 (hereafter ADAR KO), and exposure to IFNβ (100 ng/mL, 24 h).

FIG. 1 (Panel b) depicts detection of Z-RNA and A-RNA accumulation in ADAR WT or ADAR1 KO cells.

FIG. 1 (Panel c) depicts quantification of fluorescence intensity of Z-RNA and A-RNA signals in FIG. 1 (Panel b).

FIG. 1 (Panel d) depicts ADAR1 WT or KO MEFs fixed at day 7 post sgRNA transfection, exposed to the indicated nucleases, and stained for A-RNA and Z-RNA.

FIG. 1 (Panel e) depicts quantification of fluorescence intensity of Z-RNA and A-RNA signals in FIG. 1 (Panel c).

FIG. 1 (Panel f) depicts time course of Z-RNA and A-RNA formation in ADAR1 KO MEFs after IFNβ exposure.

FIG. 1 (Panel g) depicts quantification of fluorescence intensity of Z-RNA and A-RNA signals in FIG. 1 (Panel f).

FIG. 1 (Panel h) depicts Z-RNA accumulation in immortalized MEFs produced from Adar1^{-/-} mice (KO), or from or mice harboring an editing-deficient mutation point mutation (E861A), following exposure to IFNβ (100 ng/mL) for 48 hours.

FIG. 1 (Panel i) depicts quantification of fluorescence intensity of Z-RNA signal in FIG. 1 (Panel h). Unpaired Student's t-test, **p<0.005, ***p<0.0005.

FIG. 1 (Panel j) depicts 3′UTRs of inverted SINE-containing mRNAs showing Z22 enrichment (blue peaks), editing sites (red bars), location of SINEs, and location of qPCR primers used in Panel k.

FIG. 1 (Panel k) depicts qPCR analysis of the indicated inverted SINE-containing mRNA 3′UTRs following immunoprecipitation with Z22 or control IgG antibodies from ADAR1 KO MEFs stimulated with or without IFNβ (100 ng/mL) for 48 hours.

FIG. 1 (Panel l) depicts qPCR analysis of the indicated GT-repeat containing mRNAs following immunoprecipitation with Z22 or control IgG antibodies from ADAR1 KO MEFs stimulated with or without IFNβ (100 ng/mL) for 48 hours. Potential bipartite (dumbbell) Z-forming RNA structures (RNA-fold) are shown to the right of each graph, and putative Zu binding sites are outlined in pink boxes.

FIG. 1 (Panel m) depicts CD spectra of synthetic dumbbell RNA (SEQ ID NO: 11) and control RNA (SEQ ID NO: 12) at 3M NaCl. RNA structures (RNA-fold) are shown below the CD spectra grapy, and putative Zu binding sites are outlined in pink boxes.

FIG. 2 (Panel a) depicts immortalized Zbp1^{-/-} MEFs stably reconstituted with either an empty vector control (Vec) or FLAG-ZBP1 (ZBP1) subjected to CRISPR/Cas9-based control (ADAR WT) or ADAR1 ablation (ADAR KO) and growth in the presence or absence of neutralizing anti-IFNβ antibodies (0.5 μg/mL) monitored over 12 days.

FIG. 2 (Panel b) depicts photomicrographs of ADAR1 WT or ADAR KO following treatment with IFNβ (100 ng/mL) for 48 hours.

FIG. 2 (Panel c) depicts cell viability in FIG. 2 (Panel b) determined at 48 hours post IFNβ treatment.

FIG. 2 (Panel d) depicts Zbp1^{-/-} MEFs expressing FLAG-ZBP1 in which ADAR1 was present (ADAT WT) or ablated by CSISPR/Cas9 (ADAR KO) treated with indicated cytokines (100 ng/mL) and cell viability determined at 48 hours post treatment.

FIG. 2 (Panel e) depicts MEFs produced as in FIG. 2 (Panel d) treated with IFNβ or IFNγ (100 ng/mL) in the presence or absence of zVAD (50 mM) plus GSK'843 (5 mM) and viability examined at 48 hours post exposure.

FIG. 2 (Panel f) depicts proximity ligation assay showing interaction between ZBP1 and Z-RNA in IFN-treated cells when ADAR1 is ablated. Nuclei are stained with DAPI (cyan) and outlined with dashed white lines. Areas selected in the left images are shown magnified to the right of each image.

FIG. 2 (Panel g) depicts a bar graph showing quantification of PLA purple dots per cells in FIG. 2 (Panel f).

FIG. 2 (Panel h) depicts Zbp1^{-/-} MEFs reconstituted with FLAG-ZBP1 or FLAG-ZBP1DZa ablated for ADAR1 expression, treated with or without IFNβ (100 ng/mL), and FLAG immunoprecipitated subjected to RT-qPCR using primers for the 3′UTRs of Eif2ak2 or Ddx58.

FIG. 2 (Panel i) depicts FLAG-ZBP1 MEFs expressing or lacking ADAR1 exposed to IFNβ (100 ng/mL, 48 h) and anti-FLAG immunoprecipitates from these cells examined for RIPK3, MLKL and FLAG. Whole-cell extract (5% input) was examined in parallel for RIPK3, MLKL, FLAG and ADAR1 proteins.

FIG. 2 (Panel j) depicts immunofluorescence staining for pMLKL (green) in ADAR1 KO MEFs treated with IFNβ (100 ng/mL). Nuclei are stained with DAPI (blue) and outlined with dashed white lines.

FIG. 2 (Panel k) depicts a line graph representing the kinetics of pMLKL positivity, and bar graphs show the localization of pMLKL signal. pMLKL seen in the cytoplasm and translocates to the plasma membrane after IFNβ exposure in ADAR1 KO MEFs

FIG. 2 (Panel l) depicts a schematic showing ADAR1 suppresses both endogenous A-RNAs and Z-RNAs, preventing activation of PKR/MDA-5 responses downstream of A-RNA, and ZBP1-driven necroptosis downstream of Z-RNA.

FIG. 3 (Panel a) depicts structures of A-RNA, Z-RNA, B-DNA and Z-DNA.

FIG. 3 (Panel b) depicts quantification of fluorescence intensity of Z-DNA signal after treatment of MEFs with equimolar (5 μM) amounts of the indicated compounds for 18 hours.

FIG. 3 (Panel c) depicts chemical structure of CBL0137.

FIG. 3 (Panel d) depicts MEFs fixed at 12 hours post treatment with CBL0137 (5 μM) exposed to the indicated nucleases for 45 minutes, before staining for Z-DNA staining.

FIG. 3 (Panel e) depicts quantification of fluorescence intensity of Z-DNA signal in FIG. 3 (Panel d).

FIG. 3 (Panel f) depicts proportion of EREs and other repeats in the mouse genome (left), compared to distribution of Z22-enriched peaks following treatment with CBL0137 (right).

FIG. 3 (Panel g) depicts genomic distribution of L1Md T and L1Md A elements in Z22 pulldowns.

FIG. 3 (Panel h) depicts location of maximum Z-scores for L1Md T and L1Md A bound by Z22 in Panel j.

FIG. 3 (Panel i) depicts CD spectra of modified DNA [d(Cm⁸mGCACGCG)/d(CGCGTGCG)] in the presence or absence of CBL0137. Grey indicates CBL0137 alone; light blue indicates DNA alone; orange indicates addition of the 1 equivalent CBL0137 to DNA; red indicates addition of the 2 equivalent CBL0137 to DNA.

FIG. 3 (Panel j) depicts ¹⁹F NMR spectra of 8-trifluoromethyl-2′-deoxyguanosine Z-DNA d(CGC^{F}GCG)₂ in the presence or absence of CBL0137.

FIG. 3 (Panel k) depicts two views of a Molecular Dynamics model of CBL0137:Z-DNA interaction.

FIG. 4 (Panel a) depicts photomicrographs of Zbp1^{-/-} MEFs expressing Vec or FLAG-ZBP1 untreated or treated with CBL0137 (5 μM) for 18 hours.

FIG. 4 (Panel b) depicts CBL0137-induced cell death kinetics in Vec and FLAG-ZBP1 MEFs.

FIG. 4 (Panel c) depicts Vec and FLAG-ZBP1 MEFs treated with CBL0137 (5 μM) in the presence or absence of zVAD (50 mM) and GSK'843 (5 mM) and viability examined at 18 hours post treatment.

FIG. 4 (Panel d) depicts immunoblot analysis of MLKL and Caspase 3 activation in Vec and FLAG-ZBP1 MEFs.

FIG. 4 (Panel e) depicts FLAG-ZBP1 MEFs treated with CBL0137 (5 μM) for 12 hours and evaluated for ZBP1 localization and presence of Z-DNA by immunofluorescence staining. Nuclei are stained with DAPI (blue) and outlined with dashed white lines.

FIG. 4 (Panel f) depicts quantification of cells displaying co-localization of ZBP1 and Z-DNA in nucleus.

FIG. 4 (Panel g) depicts genomic distribution of Z-DNA and FLAG-ZBP1 enriched peaks after treatment with CBL0137.

FIG. 4 (Panel h) depicts Quantification of the overlap between peaks in Panel g.

FIG. 4 (Panel i) depicts percentage of repeats in each L1Base category overlapping with peaks from Panel g.

FIG. 4 (Panel j) depicts enrichment profiles for ORF1- and ORF2-intact L1Md A and L1Md T repeats overlapping with peaks from Panel g.

FIG. 4 (Panel k) depicts DNA eluted from the indicated antibodies pulldowns from CBL0137-treated/untreated FLAG-ZBP1, FLAG-ZBP1 ΔZα MEFs examined by qRT-PCR using L1Md A and T specific primers. Data were normalized to Input.

FIG. 4 (Panel l) depicts Vec and FLAG-ZBP1 MEFs treated with CBL0137 (5 μM), and anti-FLAG immunoprecipitates examined for RIPK3, MLKL and FLAG. Whole-cell extract (5% input) was examined in parallel for RIPK3, MLKL and FLAG proteins.

FIG. 4 (Panel m) depicts immunofluorescence staining for pMLKL (green) in FLAG-ZBP1 MEFs treated with CBL0137 (5 μM). Nuclei are stained with DAPI (blue) and outlined with dashed white lines.

FIG. 4 (Panel n) depicts a line graph representing the kinetics of pMLKL positivity, and bar graphs show the localization of pMLKL signal.

FIG. 4 (Panel o) depicts a 3D reconstruction of CBL0137-treated nuclei showing DNA (DAPI) herniating from gaps in the nuclear envelope (Lamin B1) (upper panels). Arrows indicate areas of herniation. Kinetics of nuclear envelope breakdown in CBL0137-treated Vec or FLAG-ZBP1 MEFs (bottom panel).

FIG. 4 (Panel p) depicts a proposed model for CBL0137-mediated nuclear necroptosis.

FIG. 5 (Panel a) depicts Kaplan-Meier overall survival curves for ZBP1 expression categories in cutaneous melanoma cases from the TCGA. Red line=high expression (expression>7^{th} empirical quartile; Q3); blue line=low expression (expression<25^{th} empirical quartile; Q1); gray line=expression between 25^{th} and 75^{th} empirical quartiles.

FIG. 5 (Panel b) depicts enrichment scores of immune and stromal cell populations in cutaneous melanoma cases (TCGA). Upper panel of heatmap shows the log₂ normalized gene expression values for Zbp1, Ripk3 and Mlkl.

FIG. 5 (Panel c) depicts violin plots of the Zbp1, Ripk3 and Mlkl expression distribution of different cell clusters from scRNA-Seq analysis from Ishikuza et al. (Ishizuka et al., Nature, 2019, 565, 43-48). Violin width indicates cell densities by expression in each population.

FIG. 5 (Panel d) depicts immunofluorescence staining for fibroblasts (green), Z-DNA (red) in B16F10 melanoma sections from CBL0137-treated/untreated WT mice at 10 days post inoculation shows infiltration of fibroblasts and infiltrated fibroblasts with Z-DNA.

FIG. 5 (Panel e) depicts quantification of fibroblasts infiltration in FIG. 5 (Panel d).

FIG. 5 (Panel f) depicts quantification of infiltrated fibroblasts with Z-DNA in FIG. 5 (Panel d).

FIG. 5 (Panel g) depicts vehicle or CBL0137 injected tumors in WT or Zbp1^{-/-} mouse stained for PDGFRa (green) or pMLKL (red). Nuclei are stained with DAPI (blue).

FIG. 5 (Panel h) depicts quantification of fibroblasts with pMLKL in FIG. 5 (Panel g).

FIG. 5 (Panel i) depicts a subcutaneous B16F10 or YUMM1.7 melanoma model established in C57BL/6 mice. For B16F10 model, at 7 days post inoculation, the mice with similar tumor volumes (50-100 mm³) received indicated treatments. For the YUMM1.7 model, at 7 days post inoculation, the mice with similar tumor volumes (100-150 mm³) received indicated treatments. The treatment was performed every 2 days for a total of four doses.

FIG. 5 (Panel j) depicts immunofluorescence staining for CD8⁺ T cells (red), CD4⁺ T (green) in B16F10 melanoma sections from WT or zbp1^{-/-} mice with indicated treatment at 11 days post inoculation shows influx of T cells.

FIG. 5 (Panel k) depicts quantification of CD8⁺ T cells in FIG. 5G.

FIG. 5 (Panel l) depicts individual tumor growth curves of syngeneic B16F10 tumor-bearing WT or zbp1^{-/-} mice treated with vehicle plus Ctrl IgG or anti-PD-1 antibody, or CBL0137 combined with Ctrl IgG or anti-PD-1 antibody.

FIG. 5 (Panel m) depicts individual tumor growth curves of syngeneic B16F10 tumor-bearing WT mice treated with vehicle plus Ctrl IgG or anti-PD-1 antibody, or CBL0137 combined with Ctrl IgG or anti-PD-1 antibody. The p-value (log-rank test) indicates a strong evidence against the null hypothesis that the risk of death is similar across these 3 categories.

FIG. 6 (Panel a) depicts time course of Z-RNA and A-RNA formation in ADAR1 WT and ADAR1 KO MEFs.

FIG. 6 (Panel b) depicts quantification of fluorescence intensity of Z-RNA and A-RNA signals in Panel a.

FIG. 6 (Panel c) depicts origin of sequenced RNA fragments in Z22 RIP-seq for ADAR1 WT cells with or without IFNβ (100 ng/ml) treatment.

FIG. 6 (Panel d) depicts editing Index for Z22 pull-downs in ADAR1 WT and KO cells before and after IFNβ treatment (100 ng/mL).

FIG. 6 (Panel e) depicts distribution of repeats showing non-random level of editing in ADAR1-WT Z22 pull-down after IFNβ treatment (Editing Index>0.5%, mean coverage per adenosine>10.

FIG. 7 (Panel a) depicts schematic of ZBP1 and its mutants.

FIG. 7 (Panel b) depicts cell viability of ZBP1 and its mutants in Panel a was determined at 48 hours post IFNβ treatment.

FIG. 7 (Panel c) depicts zbp1^{-/-} MEFs reconstituted with FLAG-ZBP1 or FLAG-ZBP1ΔZα were ablated for ADAR1 expression, treated with or without IFNβ (100 ng/mL), and FLAG or control (IgG) immunoprecipitates subjected to RT-qPCR using primers for the 3′UTRs of Xrn1, Tapbp, Slfn5 or Agl.

FIG. 7 (Panel d) depicts Lamin B1 (green) staining for nuclear envelope integrity of IFNβ (100 ng/mL) treated ADAR1 WT or ADAR1 KO MEFs at 48 hours post treatment.

FIG. 7 (Panel e) depicts kinetics of nuclear envelope breakdown in ADAR1 WT or ADAR1 KO MEFs after IFNβ (100 ng/mL) treatment. Unpaired Student's t-test, *p<0.05, **p<0.005. Scale bars represent 10 m in Panel d.

FIG. 8 (Panel a) depicts CBL0137-induced cell death kinetics in Zbp1^{-/-} MEFs stably reconstituted with empty vector (Vec), FLAG-ZBP1, or its mutants.

FIG. 8 (Panel b) depicts immunoblot analysis of MLKL activation in Zbp1^{-/-} MEFs reconstituted with empty vector (Vec), FLAG-ZBP1, or FLAG-ZBP1 mutants after CBL0137 treatment.

FIG. 8 (Panel c) depicts distribution of FLAG-enriched peaks following treatment with CBL0137 (5 μM) for 14 hours.

FIG. 8 (Panel d) depicts flow chart showing the algorithm used to construct enrichment profiles for L1Md A and L1Md T repeats.

FIG. 9 (Panel a) depicts primary MEFs, A549, HT-29, or HeLa cells were treated with CBL0137 (5 μM) for 12 hours and stained for Z-DNA.

FIG. 9 (Panel b) depicts quantification of fluorescence intensity of Z-DNA signal in Panel a.

FIG. 9 (Panel c) depicts CBL0137-induced cell death kinetics in HT-29 cells reconstituted with empty vector (Vec) or FLAG-hZBP1.

### DESCRIPTION OF EMBODIMENTS

Unless defined otherwise, all technical and scientific terms have the same meaning as is commonly understood by one of ordinary skill in the art to which the disclosed embodiments belong.

As used herein, the terms “a” or “an” mean “at least one” or “one or more” unless the context clearly indicates otherwise.

As used herein, the term “about” means that the recited numerical value is approximate and small variations would not significantly affect the practice of the disclosed embodiments. Where a numerical value is used, unless indicated otherwise by the context, “about” means the numerical value can vary by ±10% and remain within the scope of the disclosed embodiments.

As used herein, the term “alkenyl” means a straight or branched alkyl group having 2 to carbon atoms and having one or more double carbon-carbon bonds. In some embodiments, the alkenyl group has from 2 to 10 carbon atoms, from 2 to 8 carbon atoms, from 2 to 6 carbon atoms, from 2 to 4 carbon atoms, from 3 to 10 carbon atoms, from 3 to 8 carbon atoms, from 3 to 6 carbon atoms, or 3 or 4 carbon atoms. Examples of alkenyl groups include, but are not limited to, ethenyl, 1-propenyl, 2-methyl-1-propenyl, 2-propenyl, 1-butenyl, 2-butenyl, and the like.

As used herein, the term “alkoxy” means a straight or branched —O-alkyl group having 1 to 20 carbon atoms. In some embodiments, the alkoxy group has from 1 to 10 carbon atoms, from 1 to 8 carbon atoms, from 1 to 6 carbon atoms, from 1 to 4 carbon atoms, from 2 to 10 carbon atoms, from 2 to 8 carbon atoms, from 2 to 6 carbon atoms, or from 2 to 4 carbon atoms. Examples of alkoxy groups include, but are not limited to, methoxy, ethoxy, n-propoxy, isopropoxy, t-butoxy, and the like.

As used herein, the term “alkyl” means a saturated hydrocarbon group which is straight-chained or branched. In some embodiments, the alkyl group has from 1 to 20 carbon atoms, from 2 to 20 carbon atoms, from 1 to 10 carbon atoms, from 2 to 10 carbon atoms, from 1 to 8 carbon atoms, from 2 to 8 carbon atoms, from 1 to 6 carbon atoms, from 2 to 6 carbon atoms, from 1 to 4 carbon atoms, from 2 to 4 carbon atoms, from 1 to 3 carbon atoms, or 2 or 3 carbon atoms. Examples of alkyl groups include, but are not limited to, methyl (Me), ethyl (Et), propyl (e.g., n-propyl and isopropyl), butyl (e.g., n-butyl, t-butyl, isobutyl), pentyl (e.g., n-pentyl, isopentyl, neopentyl), hexyl, isohexyl, heptyl, octyl, nonyl, 4,4-dimethylpentyl, 2,2,4-trimethylpentyl, decyl, undecyl, dodecyl, 2-methyl-1-propyl, 2-methyl-2-propyl, 2-methyl-1-butyl, 3-methyl-1-butyl, 2-methyl-3-butyl, 2-methyl-1-pentyl, 2,2-dimethyl-1-propyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, and the like.

As used herein, the term “alkylamino” means an amino group substituted by an alkyl group. In some embodiments, the alkyl group is a lower alkyl group having from 1 to 6 carbon atoms. Alkylamino groups include, but are not limited to, —NHCH₂CH₃, —NH(CH₂)₂CH₃, —NH(CH₂)₃CH₃, —NH(CH₂)₄CH₃, and —NH(CH₂)₅CH₃, and the like.

As used herein, the term “alkylthio” means an —S-alkyl group having from 1 to 6 carbon atoms. Alkylthio groups include, but are not limited to, —SCH₂CH₃, —S(CH₂)₂CH₃, —S(CH₂)₃CH₃, —S(CH₂)₄CH₃, and —S(CH₂)₅CH₃, and the like.

As used herein, the term “amino” means —NH₂.

As used herein, the term “aminoalkoxy” means an alkoxy group substituted by an amino group. Examples of aminoalkoxy groups include. but are not limited to, —OCH₂NH₂, —OCH₂CH₂NH₂, —O(CH₂)₃NH₂, and —O(CH₂)₄NH₂, and the like.

As used herein, the term “aryl” means a monocyclic, bicyclic, or polycyclic (e.g., having 2, 3 or 4 fused rings) aromatic hydrocarbon. In some embodiments, the aryl group has from 6 to 20 carbon atoms or from 6 to 10 carbon atoms. Examples of aryl groups include, but are not limited to, phenyl, naphthyl, anthracenyl, phenanthrenyl, indanyl, indenyl, and tetrahydronaphthyl, and the like.

As used herein, the term “carrier” means a diluent, adjuvant, or excipient with which a compound is administered in a composition.

As used herein, the term, “compound” means all stereoisomers, tautomers, isotopes, and polymorphs of the compounds described herein.

As used herein, the terms “comprising” (and any form of comprising, such as “comprise”, “comprises”, and “comprised”), “having” (and any form of having, such as “have” and “has”), “including” (and any form of including, such as “includes” and “include”), or “containing” (and any form of containing, such as “contains” and “contain”), are inclusive and open-ended and include the options following the terms, and do not exclude additional, unrecited elements or method steps.

As used herein, the term “cyano” means —CN.

As used herein, the term “cycloalkyl” means non-aromatic cyclic hydrocarbons including cyclized alkyl, alkenyl, and alkynyl groups that have up to 20 ring-forming carbon atoms. Cycloalkyl groups have from 3 to 15 ring-forming carbon atoms, from 3 to 10 ring-forming carbon atoms, from 3 to 8 ring-forming carbon atoms, from 3 to 6 ring-forming carbon atoms, from 4 to 6 ring-forming carbon atoms, from 3 to 5 ring-forming carbon atoms, or 5 or 6 ring-forming carbon atoms. Ring-forming carbon atoms of a cycloalkyl group can be optionally substituted by oxo or sulfido. Cycloalkyl groups include, but are not limited to, monocyclic or polycyclic ring systems such as fused ring systems, bridged ring systems, and spiro ring systems. In some embodiments, polycyclic ring systems include 2, 3, or 4 fused rings. Examples of cycloalkyl groups include, but are not limited to, cyclopropyl, cyclobutyl, cyclopentyl, cyclohexyl, cycloheptyl, cyclooctyl, cyclononyl, cyclopentenyl, cyclohexenyl, cyclohexadienyl, cycloheptatrienyl, norbornyl, norpinyl, norcarnyl, adamantyl, and the like. Cycloalkyl groups can also have one or more aromatic rings fused (having a bond in common with) to the cycloalkyl ring such as, for example, benzo or thienyl derivatives of pentane, pentene, hexane, and the like (e.g., 2,3-dihydro-1H-indene-1-yl, or 1H-inden-2(3H)-one-1-yl).

As used herein, the term “halo” means halogen groups and includes, but is not limited to, fluoro, chloro, bromo, and iodo.

As used herein, the term “heteroaryl” means an aromatic heterocycle having up to 20 ring-forming atoms (e.g., C) and having at least one heteroatom ring member (ring-forming atom) such as sulfur, oxygen, or nitrogen. In some embodiments, the heteroaryl group has at least one or more heteroatom ring-forming atoms, each of which are, independently, sulfur, oxygen, or nitrogen. In some embodiments, the heteroaryl group has from 3 to 20 ring-forming atoms, from 3 to 10 ring-forming atoms, from 3 to 6 ring-forming atoms, or from 3 to 5 ring-forming atoms. In some embodiments, the heteroaryl group contains 2 to 14 carbon atoms, from 2 to 7 carbon atoms, or 5 or 6 carbon atoms. In some embodiments, the heteroaryl group has 1 to 4 heteroatoms, 1 to 3 heteroatoms, or 1 or 2 heteroatoms. Heteroaryl groups include monocyclic and polycyclic (e.g., having 2, 3 or 4 fused rings) systems. Examples of heteroaryl groups include, but are not limited to, pyridyl, pyrimidinyl, pyrazinyl, pyridazinyl, pyridinyl (including 2-aminopyridine), triazinyl, furyl, quinolyl, isoquinolyl, thienyl, imidazolyl, thiazolyl, indolyl (such as indol-3-yl), pyrryl, oxazolyl, benzofuryl, benzothienyl, pyrazolyl, benzthiazolyl, isoxazolyl, triazolyl (including 1,2,4-triazole, 1,2,3-triazole, and 5-amino-1,2,4-triazole), tetrazolyl, indazolyl, isothiazolyl, 1,2,4-thiadiazolyl, benzothienyl, purinyl, carbazolyl, isoxazolyl, benzimidazolyl, indolinyl, pyranyl, pyrazolyl, triazolyl, oxadiazolyl (including 1,2,3-oxadiazole, 1,2,4-oxadiazole, 1,2,5-oxadiazole, 3-amino-1,2,4-oxadiazole, 1,3,4-oxadiazole), thianthrenyl, indolizinyl, isoindolyl, isobenzofuranyl, pyrrolyl, benzoxazolyl, xanthenyl, 2H-pyrrolyl, 3H-indolyl, 4H-quinolizinyl, phthalazinyl, acridinyl, naphthyridinyl, quinazolinyl, phenanthridinyl, perimidinyl, phenanthrolinyl, phenazinyl, isothiazolyl, phenothiazinyl, isoxazolyl, furazanyl, phenoxazinyl groups, and the like.

As used herein, the term “hydroxy” or “hydroxyl” means an —OH group.

As used herein, the term “subject” means any animal described herein.

As used herein, the phrase “in need thereof” means that the “subject” has been identified as having a need for the particular method, prevention, or treatment. In some embodiments, the identification can be by any means of diagnosis. In any of the methods, preventions, and treatments described herein, the “subject” can be in need thereof. In some embodiments, the “subject” is in an environment or will be traveling to an environment, or has traveled to an environment in which a particular disease, disorder, or condition is prevalent.

As used herein, the term “integer” means a numerical value that is a whole number. For example, an “integer from 1 to 5” means 1, 2, 3, 4, or 5.

As used herein, the term “nitro” means —NO₂.

As used herein, the phrase “pharmaceutically acceptable” means that the compounds, materials, compositions, and/or dosage forms are within the scope of sound medical judgment and are suitable for use in contact with tissues of humans and other animals. In some embodiments, “pharmaceutically acceptable” means approved by a regulatory agency of the Federal government or a state government or listed in the U.S. Pharmacopeia or other generally recognized pharmacopeia for use in animals, and more particularly in humans. In some embodiments, the pharmaceutically acceptable compounds, materials, compositions, and/or dosage forms result in no persistent detrimental effect on the subject, or on the general health of the subject being treated. However, it will be recognized that transient effects, such as minor irritation or a “stinging” sensation, are common with administration of medicament and the existence of such transient effects is not inconsistent with the composition, formulation, or ingredient (e.g., excipient) in question.

As used herein, the phrase “pharmaceutically acceptable salt(s),” includes, but is not limited to, salts of acidic or basic groups. Compounds that are basic in nature are capable of forming a wide variety of salts with various inorganic and organic acids. Acids that may be used to prepare pharmaceutically acceptable acid addition salts of such basic compounds are those that form non-toxic acid addition salts, i.e., salts containing pharmacologically acceptable anions including, but not limited to, sulfuric, thiosulfuric, citric, maleic, acetic, oxalic, hydrochloride, hydrobromide, hydroiodide, nitrate, sulfate, bisulfate, bisulfite, phosphate, acid phosphate, isonicotinate, borate, acetate, lactate, salicylate, citrate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, bicarbonate, malonate, mesylate, esylate, napsydisylate, tosylate, besylate, orthophoshate, trifluoroacetate, and pamoate (i.e., 1,1′-methylene-bis-(2-hydroxy-3-naphthoate)) salts. Compounds that include an amino moiety may form pharmaceutically acceptable salts with various amino acids, in addition to the acids mentioned above. Compounds that are acidic in nature are capable of forming base salts with various pharmacologically acceptable cations. Examples of such salts include, but are not limited to, alkali metal or alkaline earth metal salts and, particularly, calcium, magnesium, ammonium, sodium, lithium, zinc, potassium, and iron salts. Salts also includes quaternary ammonium salts of the compounds described herein, where the compounds have one or more tertiary amine moiety.

As used herein, the terms “treat,” “treated,” or “treating” mean both therapeutic treatment and prophylactic or preventative measures wherein the object is to prevent or slow down (lessen) an undesired physiological condition, disorder or disease, or obtain beneficial or desired clinical results. For purposes herein, beneficial or desired clinical results include, but are not limited to, alleviation of symptoms; diminishment of extent of condition, disorder or disease; stabilized (i.e., not worsening) state of condition, disorder or disease; delay in onset or slowing of condition, disorder or disease progression; amelioration of the condition, disorder or disease state or remission (whether partial or total), whether detectable or undetectable; an amelioration of at least one measurable physical parameter, not necessarily discernible by the patient; or enhancement or improvement of condition, disorder or disease. Treatment includes eliciting a clinically significant response, optionally without excessive levels of side effects. Treatment also includes prolonging survival as compared to expected survival if not receiving treatment.

At various places herein, substituents of compounds may be disclosed in groups or in ranges. It is specifically intended that the disclosure include each and every individual subcombination of the members of such groups and ranges. For example, the term “C₁₋₆alkyl” is specifically intended to individually disclose methyl, ethyl, propyl, C₄alkyl, C₅alkyl, and C₆alkyl.

For compounds in which a variable appears more than once, each variable can be a different moiety chosen from the Markush group providing options for the variable. For example, where a structure is described having two R groups that are simultaneously present on the same compound, the two R groups can represent different moieties chosen from the Markush group defined for R. In another example, when an optionally multiple substituent “R” is designated in the form, for example,

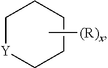

then it should be understood that substituent “R” can occur “x” number of times on the ring at any position(s), and “R” can be a different moiety at each occurrence. Further, in the above example, where the variable “Y” normally would include one or more hydrogens, such as when “Y” is CH₂, NH, etc., any H can be replaced with a substituent.

It should be appreciated that particular features of the disclosure, which are, for clarity, described in the context of separate embodiments, can also be provided in combination in a single embodiment. Conversely, various features of the disclosure which are, for brevity, described in the context of a single embodiment, can also be provided separately or in any suitable subcombination.

Appropriate compounds described herein may also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Examples of prototropic tautomers include, but are not limited to, ketone-enol pairs, amide-imidic acid pairs, lactam-lactim pairs, amide-imidic acid pairs, enamine-imine pairs, and annular forms where a proton can occupy two or more positions of a heterocyclic system including, but not limited to, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein also include hydrates and solvates, as well as anhydrous and non-solvated forms.

The compounds described herein can also include all isotopes of atoms occurring in the intermediates or final compounds. Isotopes include those atoms having the same atomic number but different mass numbers. For example, isotopes of hydrogen include tritium and deuterium. Carbon (¹²C) can be replaced at any position with ¹³C or NC. Nitrogen (¹⁴N) can be replaced with ¹⁵N. Oxygen (¹⁶O) can be replaced at any position with ⁷O or ¹⁸O. Sulfur (³²S) can be replaced with ³³S, ³⁴S or ³⁶S. Chlorine (³⁵Cl) can be replaced with ³⁷Cl. Bromine (⁷⁹Br) can be replaced with ⁸¹Br.

In some embodiments, the compounds, or salts thereof, are substantially isolated. Partial separation can include, for example, a composition enriched in any one or more of the compounds described herein. Substantial separation can include compositions containing at least about 50%, at least about 60%, at least about 70%, at least about 80%, at least about 90%, at least about 95%, at least about 97%, or at least about 99% by weight of any one or more of the compounds described herein, or salt thereof. Methods for isolating compounds and their salts are routine in the art.

Although the disclosed compounds are suitable in their present form, functional groups can be incorporated into the compounds with an expectation of similar results. In particular, thioamides and thioesters are anticipated to have very similar properties. The distance between aromatic rings can impact the geometrical pattern of the compound and this distance can be altered by incorporating aliphatic chains of varying length, which can be optionally substituted or can comprise an amino acid, a dicarboxylic acid or a diamine. The distance between and the relative orientation of monomers within the compounds can also be altered by replacing the amide bond with a surrogate having additional atoms. Thus, replacing a carbonyl group with a dicarbonyl alters the distance between the monomers and the propensity of dicarbonyl unit to adopt an anti arrangement of the two carbonyl moiety and alter the periodicity of the compound. Pyromellitic anhydride represents still another alternative to simple amide linkages which can alter the conformation and physical properties of the compound. Modern methods of solid phase organic chemistry (E. Atherton and R. C. Sheppard, Solid Phase Peptide Synthesis A Practical Approach IRL Press Oxford 1989) now allow the synthesis of homodisperse compounds with molecular weights approaching 5,000 Daltons. Other substitution patterns are equally effective.

The compounds described herein also include derivatives referred to as prodrugs, which can be prepared by modifying functional groups present in the compounds in such a way that the modifications are cleaved, either in routine manipulation or in vivo, to the parent compounds. Examples of prodrugs include compounds as described herein that contain one or more molecular moieties appended to a hydroxyl, amino, sulfhydryl, or carboxyl group of the compound, and that when administered to a patient, cleaves in vivo to form the free hydroxyl, amino, sulfhydryl, or carboxyl group, respectively. Examples of prodrugs include, but are not limited to, acetate, formate and benzoate derivatives of alcohol and amine functional groups in the compounds described herein. Preparation and use of prodrugs is discussed in T. Higuchi et al., “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are incorporated herein by reference in their entireties.

Compounds containing an amine function can also form N-oxides. A reference herein to a compound that contains an amine function also includes the N-oxide. Where a compound contains several amine functions, one or more than one nitrogen atom can be oxidized to form an N-oxide. Examples of N-oxides include N-oxides of a tertiary amine or a nitrogen atom of a nitrogen-containing heterocycle. N-Oxides can be formed by treatment of the corresponding amine with an oxidizing agent such as hydrogen peroxide or a per-acid (e.g., a peroxycarboxylic acid) (see, Advanced Organic Chemistry, by Jerry March, 4th Edition, Wiley Interscience).

Previous studies with CBL0137 focused on its ability to inhibit the FACT histone chaperone complex, activate of p53, and block NF-kappaB within tumor cells. The present disclosure describes a new function for CBL0137 on cells of the tumor mass (both tumor cells, as well as nonmalignant cells of the tumor microenvironment). This function is the induction of ZBP1-dependent necroptosis, which was not shown, or even envisioned, previously. As described herein, CBL0137, by inducing Z-DNA in cells of the tumor mass, can activate the Z-DNA sensor ZBP1. ZBP1 activated in this manner then activates necroptosis, a potently immunogenic form of cell death. It is such necroptosis that drives immunotherapy responsiveness, as nullifying necroptosis abolishes synergy with checkpoint blockade.

The present disclosure provides compounds of Formula I

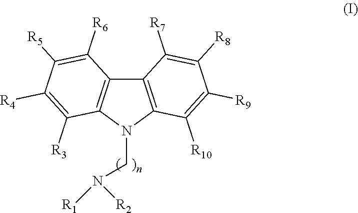

or a pharmaceutically acceptable salt thereof, wherein: each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl; each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and n is 1, 2, or 3.

In some embodiments, each of R₁ and R₂ is, independently, selected from the group consisting of hydrogen, substituted or unsubstituted alkyl, and substituted or unsubstituted aryl. In some embodiments, each of R₁ and R₂ is, independently, hydrogen or substituted or unsubstituted alkyl.

In some embodiments, each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio. In some embodiments, each of R₃, R₄, R₅, R₆, R₇, R₈, R₉, and R₁₀ is, independently, selected from the group consisting of hydrogen, halogen, hydroxyl, nitro, substituted or unsubstituted alkyl, —C(═O)alkyl, substituted or unsubstituted alkenyl, substituted or unsubstituted alkoxy, and substituted or unsubstituted amine. In some embodiments, each of R₃, R₄, R₅, R₆, R₇, R₈, R₉, and R₁₀ is, independently, selected from the group consisting of hydrogen, halogen, hydroxyl, substituted or unsubstituted alkyl, —C(═O)alkyl, and substituted or unsubstituted alkenyl. In some embodiments, R₅ and R₈ are both —C(═O)alkyl, such as —C(═O)methyl.

In some embodiments, n is 2 or 3.

In some embodiments, each of R₁ and R₂ is, independently, selected from the group consisting of hydrogen, substituted or unsubstituted alkyl, and substituted or unsubstituted aryl; each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and n is 1, 2, or 3.

In some embodiments, each of R₁ and R₂ is, independently, hydrogen or substituted or unsubstituted alkyl; each of R₃, R₄, R₅, R₆, R₇, R₈, R₉, and R₁₀ is, independently, selected from the group consisting of hydrogen, halogen, hydroxyl, nitro, substituted or unsubstituted alkyl, —C(═O)alkyl, substituted or unsubstituted alkenyl, substituted or unsubstituted alkoxy, and substituted or unsubstituted amine; and n is 2 or 3.

In some embodiments, each of R₁ and R₂ is, independently, hydrogen or substituted or unsubstituted alkyl; each of R₃, R₄, R₅, R₆, R₇, R₈, R₉, and R₁₀ is, independently, selected from the group consisting of hydrogen, halogen, hydroxyl, substituted or unsubstituted alkyl, —C(═O)alkyl, and substituted or unsubstituted alkenyl; and n is 2 or 3.

In some embodiments, each of R₁ and R₂ is, independently, hydrogen or substituted or unsubstituted alkyl; each of R₃, R₄, R₅, R₆, R₇, R₈, R₉, and R₁₀ is, independently, selected from the group consisting of hydrogen, hydroxyl, substituted or unsubstituted alkyl, and —C(═O)alkyl; and n is 2 or 3.

In some embodiments, the compound is:

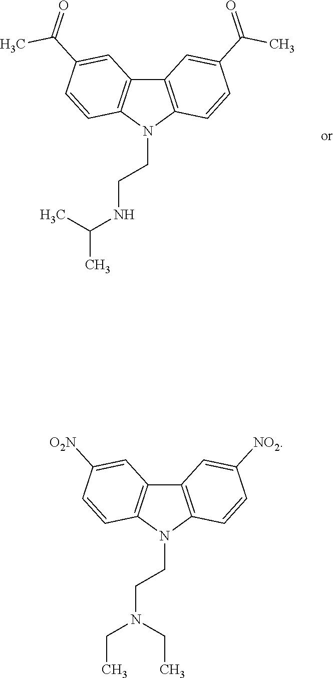 [SMILES: CC(=O)c1ccc2c(c1)c1cc(C(C)=O)ccc1n2CCNC(C)C ; CCN(CC)CCn1c2ccc([N+](=O)[O-])cc2c2cc([N+](=O)[O-])ccc21]

The present disclosure also provides compounds of Formula II:

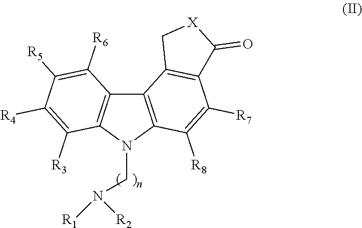

or a pharmaceutically acceptable salt thereof, wherein: each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl; each of R₃, R₄, R₅, R₆, R₇, and R₈ is, independently, selected from the group consisting of hydrogen, halogen, hydroxyl, cyano, nitro, substituted or unsubstituted alkyl, —C(═O)alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; X is substituted or unsubstituted carbon, substituted or unsubstituted nitrogen, or oxygen; and n is 1, 2, or 3.

In some embodiments, each of R₁ and R₂ is, independently, selected from the group consisting of hydrogen, substituted or unsubstituted alkyl, and substituted or unsubstituted aryl. In some embodiments, each of R₁ and R₂ is, independently, hydrogen or substituted or unsubstituted alkyl.

In some embodiments, each of R₃, R₄, R₅, R₆, R₇, and R₈ is, independently, selected from the group consisting of hydrogen, halogen, hydroxyl, cyano, nitro, substituted or unsubstituted alkyl, —C(═O)alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio. In some embodiments, each of R₃, R₄, R₅, R₆, R₇, and R₈ is, independently, selected from the group consisting of hydrogen, halogen, hydroxyl, nitro, substituted or unsubstituted alkyl, —C(═O)alkyl, substituted or unsubstituted alkenyl, substituted or unsubstituted alkoxy, and substituted or unsubstituted amine. In some embodiments, each of R₃, R₄, R₅, R₆, R₇, and R₈ is, independently, selected from the group consisting of hydrogen, halogen, hydroxyl, substituted or unsubstituted alkyl, —C(═O)alkyl, and substituted or unsubstituted alkenyl. In some embodiments, R₅ is —C(═O)alkyl, such as —C(═O)methyl.

In some embodiments, X is substituted or unsubstituted carbon, or substituted or unsubstituted nitrogen. In some embodiments, X is unsubstituted carbon or substituted nitrogen.

In some embodiments, n is 2 or 3.

In some embodiments, each of R₁ and R₂ is, independently, selected from the group consisting of hydrogen, substituted or unsubstituted alkyl, and substituted or unsubstituted aryl; each of R₃, R₄, R₅, R₆, R₇, and R₈ is, independently, selected from the group consisting of hydrogen, halogen, hydroxyl, cyano, nitro, substituted or unsubstituted alkyl, —C(═O)alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and X is substituted or unsubstituted carbon, or substituted or unsubstituted nitrogen.

In some embodiments, each of R₁ and R₂ is, independently, hydrogen or substituted or unsubstituted alkyl; each of R₃, R₄, R₅, R₆, R₇, and R₈ is, independently, selected from the group consisting of hydrogen, halogen, hydroxyl, nitro, substituted or unsubstituted alkyl, —C(═O)alkyl, substituted or unsubstituted alkenyl, substituted or unsubstituted alkoxy, and substituted or unsubstituted amine; X is unsubstituted carbon or substituted nitrogen; and n is 2 or 3.

In some embodiments, each of R₁ and R₂ is, independently, hydrogen or substituted or unsubstituted alkyl; each of R₃, R₄, R₅, R₆, R₇, and R₈ is, independently, selected from the group consisting of hydrogen, halogen, hydroxyl, substituted or unsubstituted alkyl, —C(═O)alkyl, and substituted or unsubstituted alkenyl; X is unsubstituted carbon or substituted nitrogen; and n is 2 or 3.

The present disclosure also provides compounds having Formula III:

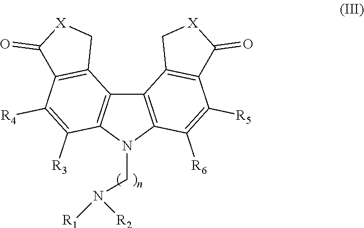

or a pharmaceutically acceptable salt thereof, wherein: each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl; each of R₃, R₄, R₅, and R₆ is, independently, selected from the group consisting of hydrogen, halogen, hydroxyl, cyano, nitro, substituted or unsubstituted alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; X is substituted or unsubstituted carbon, substituted or unsubstituted nitrogen, or oxygen; and n is 1, 2, or 3.

In some embodiments, each of R₁ and R₂ is, independently, selected from the group consisting of hydrogen, substituted or unsubstituted alkyl, and substituted or unsubstituted aryl.

In some embodiments, each of R₁ and R₂ is, independently, hydrogen, or substituted or unsubstituted alkyl.

In some embodiments, each of R₃, R₄, R₅, and R₆ is, independently, selected from the group consisting of hydrogen, halogen, hydroxyl, cyano, nitro, substituted or unsubstituted alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio. In some embodiments, each of R₃, R₄, R₅, and R₆ is, independently, selected from the group consisting of hydrogen, halogen, hydroxyl, nitro, substituted or unsubstituted alkyl, substituted or unsubstituted alkenyl, substituted or unsubstituted alkoxy, and substituted or unsubstituted amine. In some embodiments, each of R₃, R₄, R₅, and R₆ is, independently, selected from the group consisting of hydrogen, halogen, hydroxyl, and substituted or unsubstituted alkyl.

In some embodiments, X is substituted or unsubstituted carbon, or substituted or unsubstituted nitrogen. In some embodiments, X is substituted or unsubstituted carbon.

In some embodiments, n is 2 or 3.

In some embodiments, each of R₁ and R₂ is, independently, selected from the group consisting of hydrogen, substituted or unsubstituted alkyl, and substituted or unsubstituted aryl; each of R₃, R₄, R₅, and R₆ is, independently, selected from the group consisting of hydrogen, halogen, hydroxyl, cyano, nitro, substituted or unsubstituted alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and X is substituted or unsubstituted carbon, or substituted or unsubstituted nitrogen.

In some embodiments, each of R₁ and R₂ is, independently, hydrogen, or substituted or unsubstituted alkyl; each of R₃, R₄, R₅, and R₆ is, independently, selected from the group consisting of hydrogen, halogen, hydroxyl, nitro, substituted or unsubstituted alkyl, substituted or unsubstituted alkenyl, substituted or unsubstituted alkoxy, and substituted or unsubstituted amine; X is substituted or unsubstituted carbon; and n is 2 or 3.

In some embodiments, each of R₁ and R₂ is, independently, hydrogen, or substituted or unsubstituted alkyl; each of R₃, R₄, R₅, and R₆ is, independently, selected from the group consisting of hydrogen, halogen, hydroxyl, and substituted or unsubstituted alkyl; X is substituted or unsubstituted carbon; and n is 2 or 3.

The present disclosure also provides pharmaceutical compositions comprising any one or more of the compounds of Formula I, Formula II, and Formula III described herein, or pharmaceutically acceptable salts thereof, and a pharmaceutically acceptable carrier.

In any of these embodiments, the pharmaceutical composition further comprises an immune checkpoint inhibitor.

In any of these embodiments, the immune checkpoint inhibitor is selected from the group consisting of an A2AR inhibitor, a B7-H3 inhibitor, a B7-H4 inhibitor, a BTLA inhibitor, a CTLA-4 inhibitor, an IDO inhibitor, a KIR inhibitor, a LAG3 inhibitor, a NOX2 inhibitor, a PD-1 inhibitor, a PD-L1 inhibitor, a PD-L2 inhibitor, a TIM-3 inhibitor, a VISTA inhibitor, and an SIGLEC7 inhibitor, or any combination thereof.

In any of these embodiments, the immune checkpoint inhibitor is selected from the group consisting of a BTLA inhibitor, a CTLA-4 inhibitor, an IDO inhibitor, a KIR inhibitor, a LAG3 inhibitor, a NOX2 inhibitor, a PD-1 inhibitor, a PD-L1 inhibitor, a PD-L2 inhibitor, a TIM-3 inhibitor, a VISTA inhibitor, and an SIGLEC7 inhibitor, or any combination thereof.

In any of these embodiments, the immune checkpoint inhibitor is selected from the group consisting of a CTLA-4 inhibitor, a LAG3 inhibitor, a NOX2 inhibitor, a PD-1 inhibitor, a PD-L1 inhibitor, a PD-L2 inhibitor, a TIM-3 inhibitor, a VISTA inhibitor, and an SIGLEC7 inhibitor, or any combination thereof.

In any of these embodiments, the immune checkpoint inhibitor is selected from the group consisting of a CTLA-4 inhibitor, a LAG3 inhibitor, a PD-1 inhibitor, a PD-L1 inhibitor, a PD-L2 inhibitor, and a VISTA inhibitor, or any combination thereof.

In any of these embodiments, the immune checkpoint inhibitor is selected from the group consisting of a CTLA-4 inhibitor, a LAG3 inhibitor, a PD-1 inhibitor, and a VISTA inhibitor, or any combination thereof.

In any of these embodiments, the immune checkpoint inhibitor is a PD-1 inhibitor selected from the group consisting of pembrolizumab, nivolumab, cemiplimab, JTX-4014, spartalizumab, camrelizumab, sintilimab, tislelizumab, toripalimab, dostarlimab, INCMGA00012, AMP-224, and AMP-514.

In any of these embodiments, the immune checkpoint inhibitor is a CTLA-4 inhibitor selected from the group consisting of ipilimumab and tremelimumab.

In any of these embodiments, the immune checkpoint inhibitor is a LAG3 inhibitor. In some embodiments, the checkpoint inhibitor is a VISTA inhibitor.

The pharmaceutical compostions described herein can be administered to a subject in need thereof by any route of administration including, but not limited to, oral, sublingual, buccal, rectal, intranasal, inhalation, eye drops, ear drops, epidural, intracerebral, intracerebroventricular, intrathecal, epicutaneous or transdermal, subcutaneous, intradermal, intravenous, intraarterial, intraosseous infusion, intramuscular, intracardiac, intraperitoneal, intravesical infusion, and intravitreal. In some embodiments, the administration is oral, sublingual, buccal, rectal, intranasal, inhalation, eye drops, or ear drops.

In some embodiments, the carrier is a pharmaceutically acceptable carrier. Pharmaceutically acceptable carriers include, but are not limited to, aqueous vehicles such as water, alcohol (e.g., ethanol or glycol), saline solutions, dextrose solutions, and balanced salt solutions, as well as nonaqueous vehicles such as alcohols and oils, including plant or vegetable-derived oils such as olive oil, cottonseed oil, corn oil, canola oil, sesame oil, and other non-toxic oils. The compositions may also comprise one or more pharmaceutically acceptable excipients.

The pharmaceutical compositions may be formulated for administration to a subject in any suitable dosage form. The compositions may be formulated for oral, buccal, nasal, transdermal, parenteral, injectable, intravenous, subcutaneous, intramuscular, rectal, or vaginal administration. The compositions may be formulated in a suitable controlled-release vehicle, with an adjuvant, or as a depot formulation.

Preparations for parenteral administration include, but are not limited to, sterile solutions ready for injection, sterile dry soluble products ready to be combined with a solvent just prior to use, including, but not limited to, hypodermic tablets, sterile suspensions ready for injection, sterile dry insoluble products ready to be combined with a vehicle just prior to use and sterile emulsions.

Solid dosage forms include, but are not limited to, tablets, pills, powders, bulk powders, capsules, granules, and combinations thereof. Solid dosage forms may be prepared as compressed, chewable lozenges and tablets which may be enteric-coated, sugar coated or film-coated. Solid dosage forms may be hard or encased in soft gelatin, and granules and powders may be provided in non-effervescent or effervescent form. Solid dosage forms may be prepared for dissolution or suspension in a liquid or semi-liquid vehicle prior to administration. Solid dosage forms may be prepared for immediate release, controlled release, or any combination thereof. Controlled release includes, but is not limited to, delayed release, sustained release, timed pulsatile release, and location-specific pulsatile release, and combinations thereof.

Liquid dosage forms include, but are not limited to, aqueous solutions, emulsions, suspensions, solutions and/or suspensions reconstituted from non-effervescent granules and effervescent preparations reconstituted from effervescent granules. Aqueous solutions include, but are not limited to, elixirs and syrups. Emulsions may be oil-in water or water-in-oil emulsions.

Pharmaceutically acceptable excipients utilized in solid dosage forms include, but are not limited to, coatings, binders, lubricants, diluents, disintegrating agents, coloring agents, flavoring agents, preservatives, sweeteners, and wetting agents. Enteric-coated tablets, due to their enteric-coating, resist the action of stomach acid and dissolve or disintegrate in the neutral or alkaline intestines. Other examples of coatings include, but are not limited to, sugar coatings and polymer coatings. Sweetening agents are useful in the formation of chewable tablets and lozenges. Pharmaceutically acceptable excipients used in liquid dosage forms include, but are not limited to, solvents, suspending agents, dispersing agents, emulsifying agents, surfactants, emollients, coloring agents, flavoring agents, preservatives, and sweeteners.

Suitable examples of binders include, but are not limited to, glucose solution, acacia mucilage, gelatin solution, sucrose and starch paste. Suitable examples of lubricants include, but are not limited to, talc, starch, magnesium or calcium stearate, lycopodium and stearic acid. Suitable examples of diluents include, but are not limited to, lactose, sucrose, starch, kaolin, salt, mannitol and dicalcium phosphate. Suitable examples of disintegrating agents include, but are not limited to, corn starch, potato starch, bentonite, methylcellulose, agar and carboxymethylcellulose. Suitable examples of emulsifying agents include, but are not limited to, gelatin, acacia, tragacanth, bentonite, and surfactants such as polyoxyethylene sorbitan monooleate. Suitable examples of suspending agents include, but are not limited to, sodium carboxymethylcellulose, pectin, tragacanth, veegum and acacia.

Suitable examples of coloring agents include, but are not limited to, any of the approved certified water soluble FD and C dyes, mixtures thereof, and water insoluble FD and D dyes suspended on alumina hydrate. Suitable examples of sweetening agents include, but are not limited to, dextrose, sucrose, fructose, lactose, mannitol and artificial sweetening agents such as saccharin, aspartame, sucralose, acelsulfame potassium, and other artificial sweeteners. Suitable examples of flavoring agents include, but are not limited to, synthetic flavors and natural flavors extracted from plants such as fruits and mints, and synthetic blends of compounds which produce a pleasant sensation. Suitable examples of wetting agents include, but are not limited to, propylene glycol monostearate, sorbitan monooleate, diethylene glycol monolaurate and polyoxyethylene laural ether. Suitable examples of enteric-coatings include, but are not limited to, fatty acids, fats, waxes, shellac, ammoniated shellac and cellulose acetate phthalates. Suitable examples of film coatings include, but are not limited to, hydroxyethylcellulose, sodium carboxymethylcellulose, polyethylene glycol 4000 and cellulose acetate phthalate. Suitable examples of preservatives include, but are not limited to, glycerin, methyl and propylparaben, ethylparaben, butylparaben, isobutylparaben, isopropylparaben, benzylparaben, citrate, benzoic acid, sodium benzoate and alcohol.

Suitable examples of elixirs include, but are not limited to, clear, sweetened, hydroalcoholic preparations. Pharmaceutically acceptable carriers used in elixirs include solvents. Suitable examples of syrups include, but are not limited to, concentrated aqueous solutions of a sugar, for example, sucrose, and may contain a preservative. An emulsion is a two-phase system in which one liquid is dispersed throughout another liquid.

Pharmaceutically acceptable carriers used in emulsions can also include emulsifying agents and preservatives. Suspensions may use pharmaceutically acceptable suspending agents and preservatives. Pharmaceutically acceptable substances used in non-effervescent granules, to be reconstituted into a liquid oral dosage form, include, but are not limited to, diluents, sweeteners, and wetting agents. Pharmaceutically acceptable substances used in effervescent granules, to be reconstituted into a liquid oral dosage form, include, but are not limited to, organic acids and a source of carbon dioxide. Sources of carbon dioxide include, but are not limited to, sodium bicarbonate and sodium carbonate. Coloring and flavoring agents may be used in all such dosage forms.

Additional excipients that may be included in any dosage forms include, but are not limited to, antimicrobial agents, isotonic agents, buffers, antioxidants, local anesthetic agents, sequestering or chelating agents, analgesic agents, antiemetic agents, and other agents to enhance selected characteristics of the formulation.

In some embodiments, the ratio of the compound having Formula I to the checkpoint inhibitor in the pharmaceutical composition is from about 0.01:1 to about 100:1 w/w. In some embodiments, the ratio of the compound having Formula II to the checkpoint inhibitor in the pharmaceutical composition is from about 0.01:1 to about 100:1 w/w. In some embodiments, the ratio of the compound having Formula III to the checkpoint inhibitor in the pharmaceutical composition is from about 0.01:1 to about 100:1 w/w.

The present disclosure also provides methods of treating cancer in a subject comprising administering to the subject in need thereof a compound of Formula I, of Formula II, or of Formula III, or pharmaceutically acceptable salt thereof, or a composition comprising the same, and an immune checkpoint inhibitor. Any of the compounds of Formula I, of Formula II, or of Formula III described herein can be used. Any of the immune checkpoint inhibitors described herein can be used. The compositions used to treat a subject may comprise: i) a combination of a compound of Formula I, of Formula II, or of Formula III, or pharmaceutically acceptable salt thereof, and an immune checkpoint inhibitor; ii) a compound of Formula I, of Formula II, or of Formula III, or pharmaceutically acceptable salt thereof; or iii) an immune checkpoint inhibitor. In some embodiments, separate compositions ii) and iii) can be administered to a subject.

In any of these embodiments, the cancer is selected from the group consisting of melanoma, bladder cancer, renal cancer, colon cancer, head and neck cancer, gastric cancer, lung cancer, and pancreatic cancer. In some embodiments, the cancer is melanoma. In some embodiments, the cancer is bladder cancer. In some embodiments, the cancer is renal cancer. In some embodiments, the renal cancer is renal cell carcinoma. In some embodiments, the cancer is colon cancer. In some embodiments, the colon cancer is an MSI-hi tumor. In some embodiments, the cancer is head and neck cancer. In some embodiments, the cancer is gastric cancer. In some embodiments, the cancer is lung cancer. In some embodiments, the cancer is pancreatic cancer. In some embodiments, the pancreatic cancer is pancreatic adenocarcinoma.

In some embodiments, the compound of Formula I, Formula II, or Formula III is administered before the immune checkpoint inhibitor. In some embodiments, the compound of Formula I, Formula II, or Formula III is administered after the immune checkpoint inhibitor. In some embodiments, the compound of Formula I, Formula II, or Formula III is administered in the same composition as the immune checkpoint inhibitor.

The amount of the compound having Formula I, Formula II, or Formula III to be administered may be that amount which is therapeutically effective. The dosage to be administered may depend on the characteristics of the subject being treated, e.g., the particular animal treated, age, weight, health, types of concurrent treatment, if any, and frequency of treatments, and on the nature and extent of the cancer, and can be easily determined by one skilled in the art (e.g., by the clinician). The selection of the specific dose regimen can be selected or adjusted or titrated by the clinician according to methods known to the clinician to obtain the desired clinical response. In addition, in vitro or in vivo assays may optionally be employed to help identify optimal dosage ranges. The precise dose to be employed in the compositions may also depend on the route of administration, and should be decided according to the judgment of the practitioner and each patient's circumstances.

The compositions may be prepared to provide from about 0.05 mg to about 500 mg of the compound having Formula I, Formula II, or Formula III, or pharmaceutically acceptable salt thereof. In some embodiments, the compositions may comprise from about 1 mg to about 200 mg, from about 10 mg to about 200 mg, from about 10 mg to about 100 mg, from about 50 mg to about 100 mg, from about 20 mg to about 400 mg, from about 100 mg to about 300 mg, or from about 50 mg to about 250 mg of the compound of Formula I, Formula II, or Formula III, or an isomer, tautomer, or solvate thereof, or a pharmaceutically acceptable salt thereof.

Suitable dosage ranges for oral administration include, but are not limited to, from about 0.001 mg/kg body weight to about 200 mg/kg body weight, from about 0.01 mg/kg body weight to about 100 mg/kg body weight, from about 0.01 mg/kg body weight to about 70 mg/kg body weight, from about 0.1 mg/kg body weight to about 50 mg/kg body weight, from 0.5 mg/kg body weight to about 20 mg/kg body weight, or from about 1 mg/kg body weight to about 10 mg/kg body weight. In some embodiments, the oral dose is about 5 mg/kg body weight.

Suitable dosage ranges for intravenous administration include, but are not limited to, from about 0.01 mg/kg body weight to about 500 mg/kg body weight, from about 0.1 mg/kg body weight to about 100 mg/kg body weight, from about 1 mg/kg body weight to about 50 mg/kg body weight, or from about 10 mg/kg body weight to about 35 mg/kg body weight.

Suitable dosage ranges for other routes of administration can be calculated based on the forgoing dosages as known by one skilled in the art. For example, recommended dosages for intradermal, intramuscular, intraperitoneal, subcutaneous, epidural, sublingual, intracerebral, transdermal, or inhalation are in the range from about 0.001 mg/kg body weight to about 200 mg/kg body weight, from about 0.01 mg/kg body weight to about 100 mg/kg body weight, from about 0.1 mg/kg body weight to about 50 mg/kg body weight, or from about 1 mg/kg body weight to about 20 mg/kg body weight. Effective doses may be extrapolated from dose-response curves derived from in vitro or animal model test systems. Such animal models and systems are well known in the art.

In some embodiments, the amount of the compound administered to the subject is from about 0.1 mg to about 500 mg, from about 1 mg to about 250 mg, from about 5 mg to about 400 mg, from about 10 mg to about 250 mg, from about 20 mg to about 200 mg, or from about 40 mg to about 100 mg. In some embodiments, the amount of the compound administered to the subject is from about 0.1 mg to about 500 mg. In some embodiments, the amount of the compound administered to the subject is from about 1 mg to about 250 mg. In some embodiments, the amount of the compound administered to the subject is from about 5 mg to about 400 mg. In some embodiments, the amount of the compound administered to the subject is from about 10 mg to about 250 mg. In some embodiments, the amount of the compound administered to the subject is from about 20 mg to about 200 mg. In some embodiments, the amount of the compound administered to the subject is from about 40 mg to about 100 mg.

The present disclosure also provides uses of any one or more of the compounds having Formula I, Formula II, or Formula III, or pharmaceutically acceptable salts thereof, or compositions comprising any one or more of the compounds having Formula I, Formula II, or Formula III, or a pharmaceutically acceptable salts thereof, and an immune checkpoint inhibitor in the preparation of a medicament for treating a subject having cancer.

The present disclosure also provides uses of any one or more of the compounds having Formula I, Formula II, or Formula III, or pharmaceutically acceptable salts thereof, or compositions comprising any one or more of the compounds having Formula I, Formula II, or Formula III, or pharmaceutically acceptable salts thereof, and an immune checkpoint inhibitor for treating a subject having cancer.

The present disclosure also provides methods of identifying a compound that induces necroptosis. The methods comprise contacting a cell deficient in Adenosine Deaminase RNA Specific 1 (ADAR1) polypeptide with the compound. An increase in cell death indicates that the compound induces necroptosis. In some embodiments, the cell is a murine embryo fibroblast (MEF) from Adar-deficient mice. In some embodiments, the cell is an ADAR1 knock-out cell, where Adar is ablated by CRISPR-based or similar approaches. In some embodiments, the cell comprises a functional Z-form nucleic acid Binding Protein 1 (ZBP1) polypeptide.

The present disclosure also provides methods of identifying a compound that induces Z-DNA formation in vitro. The methods comprise contacting a double-stranded GC-rich B-DNA oligonucleotide incorporating 2′-O-methyl-8-methyl modification of internal guanosine nucleosides (m⁸Gm) with the compound. An increase in the formation of Z-DNA indicates that the compound induces Z-DNA formation. In some embodiments, the oligonucleotide is an octamer.

The present disclosure also provides methods of identifying a compound that induces Z-DNA formation in a cell. The methods comprise contacting a live cultured cell with the compound, and detecting Z-DNA formation. An increase in formation of Z-DNA within the cell indicates that the compound induces Z-DNA formation. In some embodiments, the cell is a cell line. In some embodiments, the cell is a eukaryotic cell. In some embodiments, the cell is a MEF from Adar-deficient mice. In some embodiments, the cell is an ADAR1 knock-out cell, where Adar is ablated by CRISPR-based or similar approaches. In some embodiments, the cell comprises a functional Z-form nucleic acid Binding Protein 1 (ZBP1) polypeptide. An increase in cell death indicates that the compound induces ZBP1-dependent necroptosis.

In any of the embodiments described herein, the Z-DNA is detected by immunodetection, such as by immunoprecipitation or immunofluorescence. In some embodiments, the immunodetection can be carried out by an antibody that specifically binds to the Z-form of DNA. In some embodiments, the Z-DNA is detected by ¹⁹F NMR or circular dichroism.

In order that the subject matter disclosed herein may be more efficiently understood, examples are provided below. It should be understood that these examples are for illustrative purposes only and are not to be construed as limiting the claimed subject matter in any manner.

### EXAMPLES

### Example 1: General Methods

### Mice

Six- to eight-week-old female wild-type C57BL/6 mice were obtained from the Jackson Laboratory. Prior to all experiments, purchased mice were allowed one week to acclimate to housing conditions at the Fox Chase Cancer Center. All experimental mice were housed under specific pathogen-free conditions and all in vivo experiments were conducted under protocols approved by the Committee on Use and Care of Animals at the Fox Chase Cancer Center.

### Cell Lines

Primary and Immortalized MEFs B16-F10 (ATCC, CRL-6475), YUMMER 1.7, cells were cultivated in RPMI-1640 supplemented with 10% fetal bovine serum and 1×penicillin and streptomycin. YUMMER 1.7 cells were cultured in DMEM/F12 supplemented with 10% fetal bovine serum, 1×penicillin and streptomycin and 1× non-essential amino acids. MEFs were maintained in DMEM supplemented with 15% FBS, 1 mM sodium pyruvate, 1× GlutaMAX, and 1% penicillin/streptomycin. All cells were cultured at 37° C., 5% CO₂.

### Immunofluorescence Microscopy

For immunofluorescent staining of cultured cells, cells were plated on 8-well glass slides (EMD Millipore), and allowed to adhere for at least 24 hours before use in experiments. Following treatment, cells were fixed with freshly-prepared 4% (w/v) paraformaldehyde, permeabilized in 0.5% (v/v) Triton X-100, blocked with MAXblock™ Blocking Medium (Active Motif), and incubated overnight with primary antibodies at 4° C. After three washes in PBS, slides were incubated with fluorophore-conjugated secondary antibodies for 1 hour at room temperature. Following an additional three washes in PBS, slides were mounted in ProLong Gold antifade reagent (Thermo Fisher Scientific) and imaged by confocal microscopy on a Leica SP8 instrument. For immunofluorescent staining of tumor frozen sections, frozen tumor sections were cut at 50 μm in a cryostat microtome. Sections were permeabilized with 0.5% (v/v) Triton X-100 in PBS and blocked with MAXblock™ Blocking Medium (Active Motif), and incubated overnight with primary antibodies at 4° C. After three washes in PBS, slides were incubated with fluorophore-conjugated secondary antibodies for 1 hour at room temperature. Following an additional three washes in PBS, slides were mounted in ProLong Gold antifade reagent (Thermo Fisher Scientific) and imaged by confocal microscopy on a Leica SP8 instrument. Fluorescence intensity was quantified using Leica LAS X software. When required, RNase A (1 mg/mL) or DNase I (25 U/mL) was used for 1 hour at 37° C. before primary antibody incubation. Primary antibodies were used for immuno-fluorescence studies: Z-NA (Z22, Absolute Antibody), A-RNA (9D5, Millipore), phosphorylated murine MLKL (Cat. 37333, Cell Signaling), FLAG (Cat.A00187, GenScript), lamin B1 (ab16048, Abcam), PDGFRa (Cat. 14-1401-82, Thermo Fisher Scientific), CD3 (eBioscience), CD8 (ebioscience).

### ADAR1 Gene Deletion by CRISPR/Cas9

Immortalized Zbp1^{-/-} stably reconstituted with FLAG-ZBP1 MEFs were transiently transfected with TrueGuide Synthetic sgRNA against mouse Adar1 (ThermoFisher Scientific, CRISPR162007 SGM) and TrueCut Cas9 Protein (ThermoFisher Scientific, A36499) by Lipofectamine CRISPRMAX Cas9 Transfection Reagent (ThermoFisher Scientific, CMAX00008). After two days, cells were harvest and examined ADAR1 protein level by western blot.

### Duolink Proximity Ligation In Situ Assay

Cells were fixed with freshly prepared 4% (w/v) paraformaldehyde for 10 minutes, permeabilized in 0.5% (v/v) Triton X-100 for 15 minutes, and subjected to Duolink In Situ assay according to the manufacturer's instructions (Sigma-Aldrich, Duolink™ In Situ Red Starter Kit Mouse/Rabbit).

### RNA Immunoprecipitation (RIP)-Seq

Wild-type Adar1 or knockout Adar1 immortalized Zbp1^{-/-} MEFs stably reconstituted with FLAG-ZBP1 were treated with/without IFNβ for 72 hours, and then harvested. RNA immunoprecipitation (RIP) assays were conducted using the Magna RIP RNA-Binding Protein Immunoprecipitation Kit (Millipore) following the manufacturer's instructions. Briefly, cell pellets were lysed in RIP lysis buffer, following incubation with RIP Buffer containing magnetic beads conjugated with Z-RNA or isotype control antibody at 4° C. overnight. Then, samples were incubated with proteinase K and immunoprecipitated RNAs were recovered by phenol:chloroform:isoarnyl alcohol. Paired-end RNA-sequencing was performed by Novogene.

### Chromatin Immunoprecipitation (ChIP)-Seq

Immortalized Zbp1^{-/-} MEFs were treated with/without CBL0137, then harvested. ChIP were performed using EZ-Magna ChIP HiSens Chromatin Immunoprecipitation Kit (Millipore) following the manufacturer's instructions. Briefly, cells were cross-linked by 1% formaldehyde. Nuclei were isolated by Nuclei Isolation Buffer. The cross-linked DNA were shear to 200-500 base pair in length via sonication. Then, the sheared DNA was immunoprecipitated using anti-Z-DNA, FLAG or isotype control antibody at 4° C. overnight. Finally, Paired-end 150 DNA sequencing was performed by Novogene.

### Quantitative PCR

RNA from RIP was reverse transcribed into cDNA using SuperScript IV VILO Master Mix (Thermo Fisher Scientific). cDNA or DNA from ChIP was used as template and quantitative PCR was performed using SYBR Green (Thermo Fisher Scientific). Primers were listed below:

| | Eif2ak2-fwd: |
|---|---|
| (SEQ ID NO: 1) | |
| | AGCTCCAAATAACCAAGATAC; |
| | Eif2ak2-rev: |
| (SEQ ID NO: 2) | |
| | CTCTGCTCTACACTCTATCTCC; |
| | Ddx58-fwd: |
| (SEQ ID NO: 3) | |
| | GAATGCACTCTGTAGTCCAG; |
| | Ddx58-rev: |
| (SEQ ID NO: 4) | |
| | ATAAATGAAAGTCAGCTCTCAG; |
| | Ifih1-fwd: |
| (SEQ ID NO: 5) | |
| | GGAATGCCCATGAGGTATTG; |
| | Ifih1-rev: |
| (SEQ ID NO: 6) | |
| | AGCTTGCCACATTGCATTG; |
| | L1Md A-fwd: |
| (SEQ ID NO: 7) | |
| | ACATAGGGAAGCAGGCTACCC; |
| | L1Md A-rev: |
| (SEQ ID NO: 8) | |
| | GGCAAGACTCTGCTGGCAAGG; |
| | L1Md G-fwd: |
| (SEQ ID NO: 9) | |
| | AAGCACAGAGGCGCTGAGGCAG; |
| | L1Md G-rev: |
| (SEQ ID NO: 10) | |
| | GACTAATTTCCTAAGTTCGGC. |

### Immunoprecipitation and Immunoblotting

Immortalized Zbp1^{-/-} MEFs stably reconstituted with FLAG-tagged WT or empty vector were lysed in IP lysis buffer (Thermo Fisher Scientific, cat #87787) supplemented with protease and phosphatase inhibitor (Thermo Fisher Scientific, cat #78444). Cell lysates were incubated on ice for 10 minutes, and briefly sonicated to shear chromatin, then cleared by high-speed centrifugation (20,000 g, 10 minutes) at 4° C. After saving 5% of the total cell lysate for input, the extracts were subjected to immunoprecipitation with anti-FLAG M2 affinity gel, according to the manufacturer's instructions (Sigma, cat #FLAGIPTT). Resin was eluted with 3×FLAG peptide and the supernatants subjected to immunoblot analysis as described before 61. Primary antibodies were used at the following dilutions: ADAR1 (1:1000), phosphorylated murine MLKL (1:2000), total MLKL (1:2000), RIPK3 (1:2000), FLAG (1:2000), GAPDH (1:4000).

### TCGA Data Analysis.

RSEM normalized gene expression for melanoma (TCGA-SKCM) (PMID: 26091043) was obtained from Broad Institute Firehose pipeline (Data version 2016_01_28). This data was log2 normalized (RSEM expression value+1) was stratified into 3 classes based on quartiles where cases with expression>7^{th} empirical quartile (Q3) were classified as high-expressors and cases with expression<25^{th} empirical quartile (Q1) were classified as low-expressors, while cases that are between these two ranges were classified as no-change in expression category. Survival curves for these categories were compared with log-rank tests, and these calculations were done using the R ‘survival’ package (Therneau, T. M. & Grambsch, P. M. Modeling Survival Data: Extending the Cox Model (Springer-Verlag, 2010)).

### Tumor Infiltrating Cell Population Analysis

Microenvironment Cell Populations-counter analysis (PMID: 27765066) was used to estimate the tumor infiltrating populations. Log2 transformed data was used as input. Heatmaps were plotted using pheatmap package available in Bioconductor (https://cran.r-project.org/web/packages/pheatmap/index.html). All calculations were done in R programming environment.

scRNA-Seq Data Analysis.

The scRNA-seq data from Ishizuka et al. study (GSE110746) was used. The data was analyzed as described in Gabitova-Comell et al. (PMID: 32976774). Briefly, the data was analyzed using Cell Ranger analysis pipeline (v1.2). For all the downstream analyses and violin plots were done using Seurat package (PMID: 25867923). The clusters were classified based on Ishizuka et al. study.

### Mouse Melanoma Models

Mice were anesthetized, shaved at the injection site, and then injected in the flank subcutaneously with 5×10⁵ B16-F10 cells and 2×10⁶ YUMM1.7 cells. Tumors were measured every two days once palpable with a caliper. Tumor volume was calculated using the volume formula: 0.5×D×d² where D is the longer diameter and d is the shorter diameter. Treatment was initiated when mean tumor size was 50-100 mm³ for B16F10 tumor or 100-150 mm³ for YUMM1.7 tumor. 50 μl CBL0137 or vehicle was delivered via intratumor injection and 200 μg anti-PD-1 antibody (Bio X cell, clone RMP1.14) or isotype control IgG (Bio X cell, clone 2A3) was delivered via intraperitoneal injection. Treatment was performed every two days for a total of 4 doses. Mice were euthanized when tumors reached endpoints (volume greater than or equal to 2000 mm³ for B16F10 tumor or 1000 mm³ for YUMM1.7 tumor) or upon ulceration/bleeding. Survival analyses reflect this endpoint.

### Statistics.

Statistical significance was determined by use of either unpaired Student's t-test for comparison between two groups or two-way ANOVA followed by Tukey's test for comparisons between multiple (>2) groups. P-values of 0.05 or lower were considered significant. Graphs were generated using GraphPad Prism 6.0 software.

### Example 2: ADAR1 Loss Triggers Z-RNA Accumulation

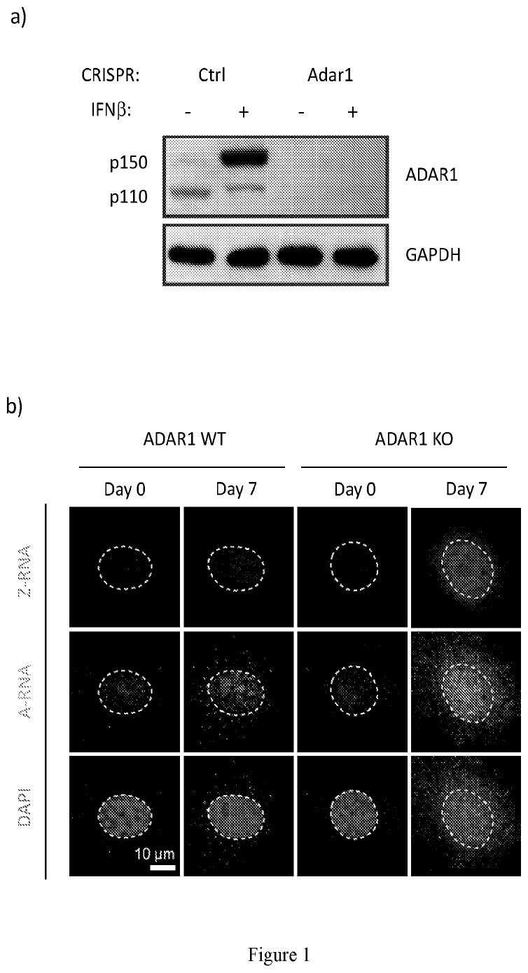

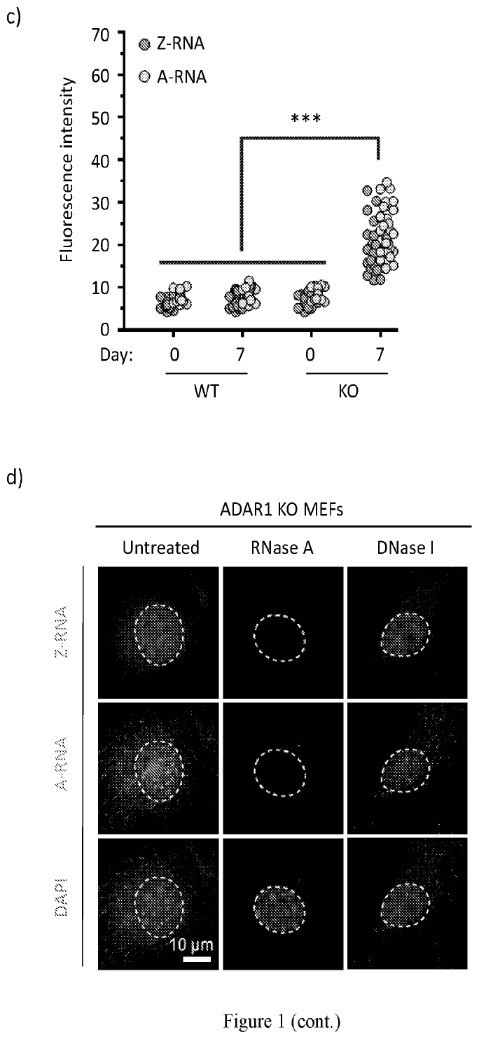

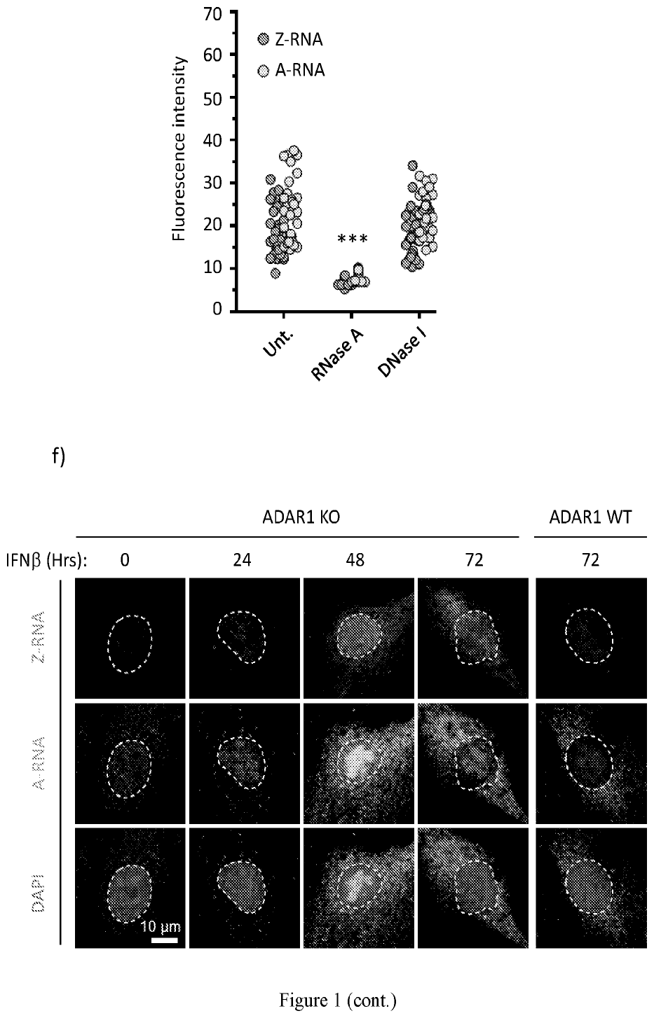

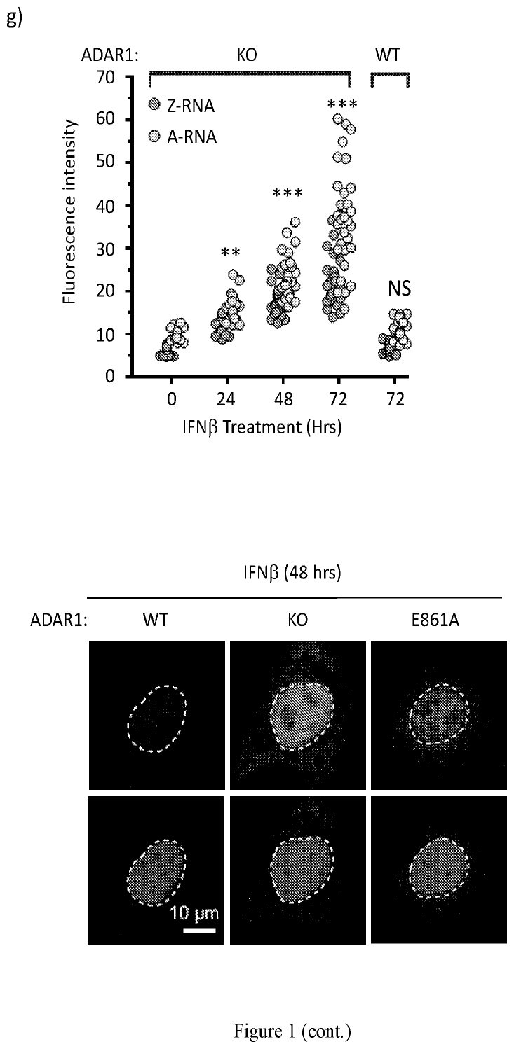

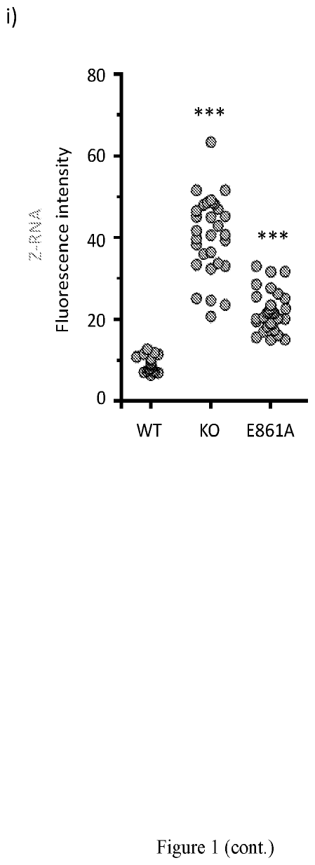

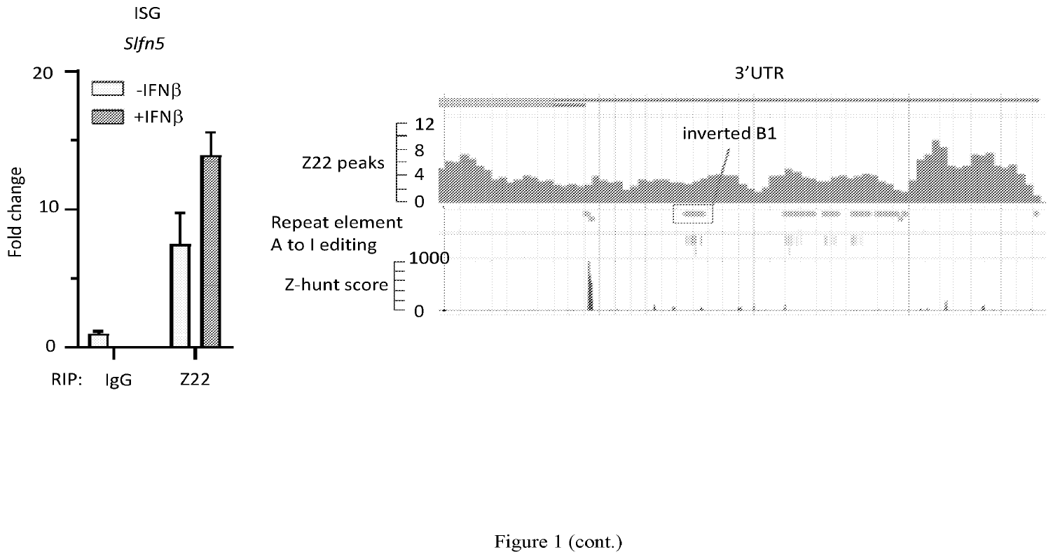

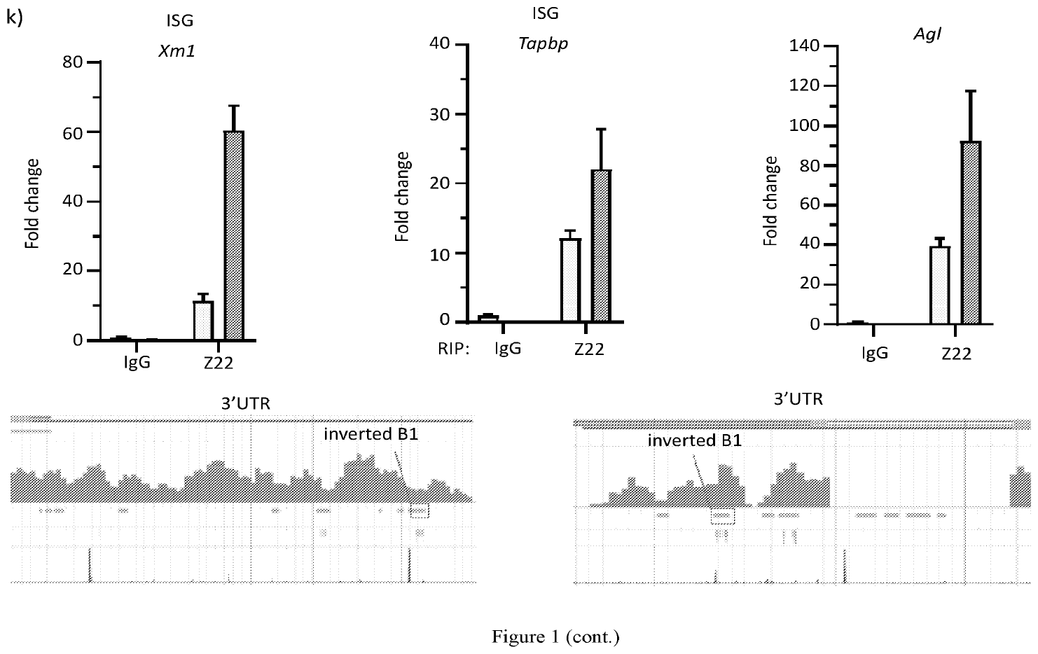

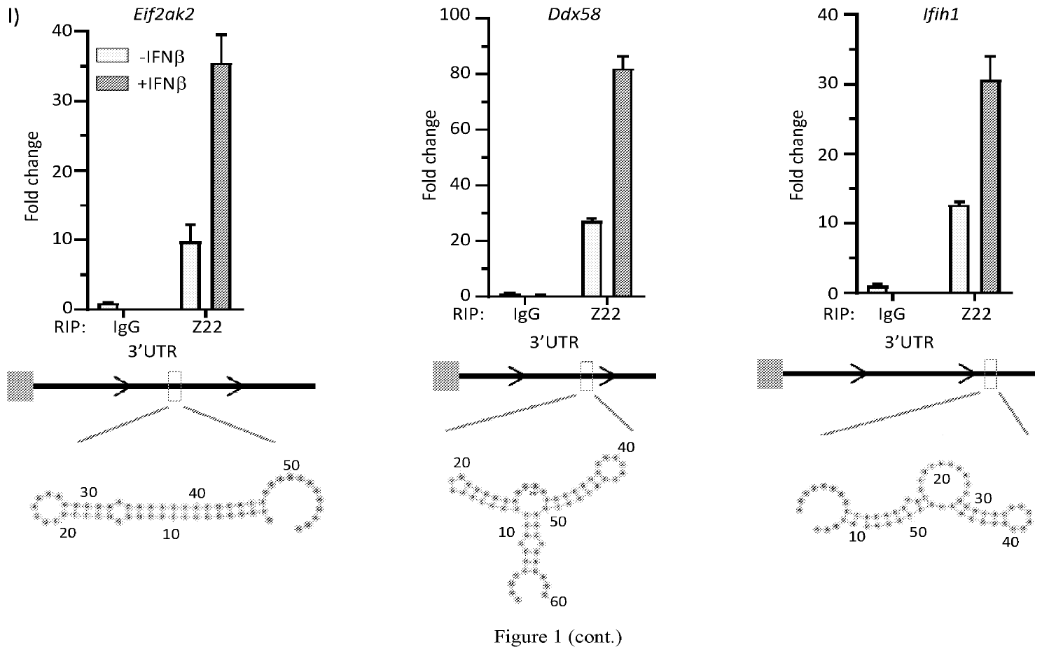

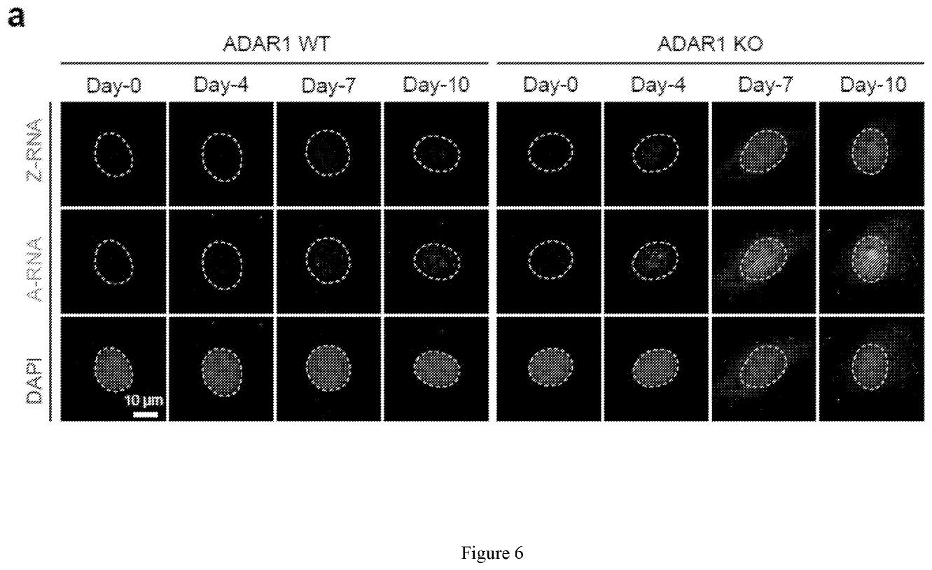

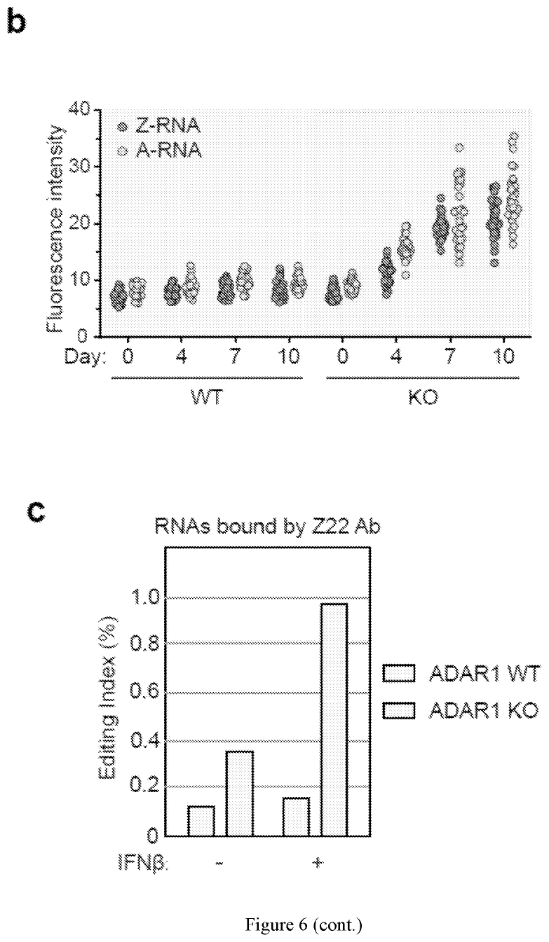

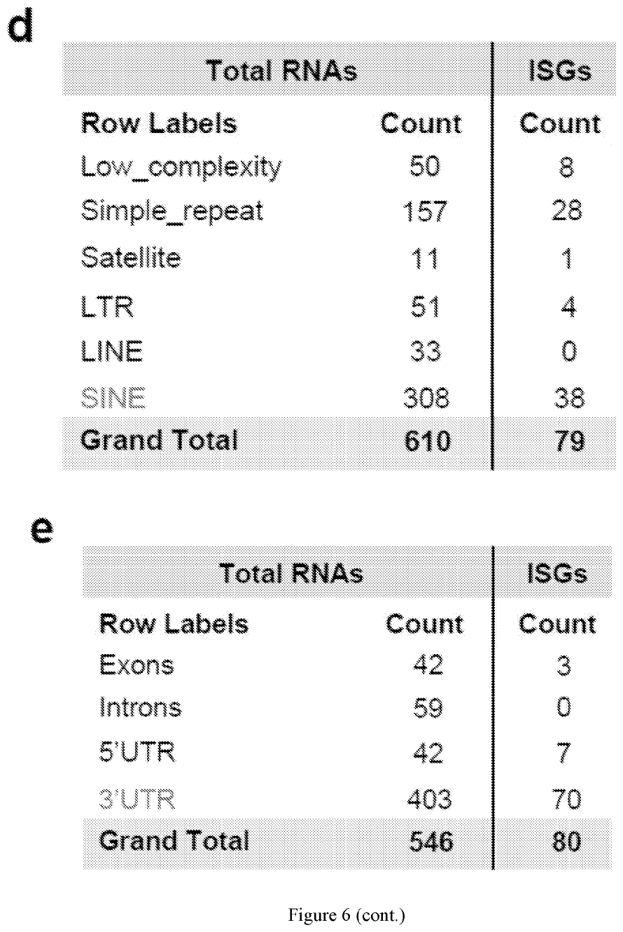

To test if ADAR1 repressed production of endogenous Z-RNAs, we ablated Adar1 expression by CRISPR/Cas9 approaches in immortalized wild-type (WT) murine embryo fibroblasts (MEFs) and confirmed that these cells lost basal expression of both ADAR1 p110 and p150 isoforms, as well as type I IFN-induced expression of the ADAR1 p150 isoform (see, FIG. 1, Panel a). We next cultured control ADAR1 WT and ADAR1-deficient (ADAR1 KO) MEFs over a 10 day period, periodically fixing the cells in formaldehyde and examining them for the presence of Z-RNA by an immunofluorescence-based assay using an antibody (clone Z22) originally raised to Z-DNA, but found by us and others to also detect Z-RNA in vitro (Hardin et al., Biochemistry, 1987, 26, 5191-5199) and in cellulo (Zhang et al., Cell, 2020, 180, 1115-1129 e1113). Cells lacking ADAR1 manifested a predominantly nuclear signal when stained with the Z22 antibody, detectable by day 4 post ADAR1-ablation and increasing in intensity over 10 days. In contrast, similarly-cultured control MEFs expressing wild-type ADAR1 did not develop a detectable signal (see, FIG. 1, Panel b and Panel c and FIG. 6, Panel a and Panel b). The signal produced by the Z22 antibody in ADAR1-deficient cells was sensitive to RNase A but not to DNase 1, strongly suggesting that it originated from the accumulation of endogenous Z-RNAs (see, FIG. 1, Panel d and Panel e). Notably, ADAR1-deficient cells also showed robust accumulation of A-RNA, with kinetics of induction largely paralleling that of Z-RNA (see, FIG. 1, Panel b, Panel c, Panel d, and Panel e; and FIG. 6, Panel a and Panel b). Together, these results indicate that ADAR1, besides quenching cellular A-RNA, prevents the accumulation of endogenous Z-RNA.

Type I IFNs (such as IFNβ) stimulate ERE transcription and dsRNA induction in other contexts (Liu et al., Nat Med, 2019, 25, 95-102; Canadas et al., Nat Med, 2018, 24, 1143-1150; and Chuong et al., Science, 2016, 351, 1083-1087), suggesting that these cytokines might also boost Z-RNA levels in ADAR1-deficient cells. Indeed, we found that exposure of ADAR1 KO MEFs to IFNβ strongly stimulated production of both Z-RNA and A-RNA within 48 hours of treatment (see, FIG. 1, Panel f and Panel g). By stably reconstituting MEFs from Adar1^{-/-} mice with a Za-domain deletion (ΔZα), or by employing MEFs from ADAR1 editing null (E861A) knock-in mice (see, FIG. 6, Panel c), treating these cells with IFNβ, and then examining Z-RNA accumulation, we found that ADAR1 repressed endogenous Z-RNAs in a manner requiring both the Za domain and editing activity (see, FIG. 1, Panel h and Panel i).

To identify endogenous Z-RNAs repressed by ADAR1, we used the Z22 antibody to immuno-precipitate Z-RNAs from ADAR1 WT or ADAR1 KO MEFs following IFNβ treatment, and sequenced these RNAs. We then assessed editing in these RNA sequences by calculating their editing index (Bazak et al., Nucleic Acids Res, 2014, 42, 6876-6884), a robust, normalized, and quantitative measure of A→1 modification. In the absence of IFNβ treatment, Z22-associated RNAs displayed an editing index of ˜0.1% (see, FIG. 6, Panel c), occurring more frequently than other transitions or transversions (which were found at ˜0.06% in our dataset). As ADAR1 p150 levels are typically very low in unstimulated MEFs, and as RNAs from both ADAR1 WT and ADAR1 KO manifested similar basal editing indices (see, FIG. 6, Panel c), the low basal level of editing likely results from the activity of ADAR2. RNAs in Z22 pulldowns from IFNβ-exposed ADAR1 WT MEFs, however, displayed a significantly increased editing index compared to either untreated ADAR1 WT cells or IFNβ-treated ADAR1 KO MEFs (see, FIG. 6, Panel c); this increase in the editing index in therefore attributable to IFN-induced expression of the ADAR1 p150 isoform, its binding of Z-RNA through the Zu domain (which ADAR1 p110 and ADAR2 lack), and the consequent editing of these Z-RNAs. Most of the ADAR1-edited RNAs pulled down by the Z22 antibody were SINEs and simple repeats, and a notable fraction of these were found in ISG-encoded mRNA transcripts (see, FIG. 6, Panel d). There was no significant enrichment of either intronic RNAs (see, FIG. 6, Panel e). When we examined mRNA sequences in Z22 pulldowns for the location of editing by ADAR1 p150, we found that edits mostly occurred in their 3′UTRs (see, FIG. 6, Panel f). Interestingly, the 3′UTRs of ISG mRNAs were disproportionately targeted for editing by ADAR1 p150. We selected three ISG mRNAs with high editing indices (Xrn1, Tapbp, Z), and found that these transcripts harbored inverted SINEs in their 3′UTRs, with A→G edits clustered in the vicinity of these SINEs (see, FIG. 1, Panel j). Each of these 3′UTRs were robustly pulled down in an IFN-dependent manner by Z22, but not by a control IgG, from ADAR1 KO lysates (see, FIG. 1, Panel h). Thus, 3′UTRs containing inverted SINEs represent one class of ADAR1-repressed endogenous Z-RNAs. Notably, these elements also form A-RNAs following ADAR1 loss (Ishizuka et al., Nature, 2019, 565, 43-48). Interestingly, only a subset of Z22-enriched mRNAs had SINEs in their 3′UTRs. Many of the 3′UTRs not harboring SINEs were, however, rich in Z-prone GT repeats (Nichols et al., Nature Communications, 2021, 12, 793). For example, the ISG mRNAs encoding the dsRNA sensors PKR (Eif2ak2), RIG-I (Ddx58) and MDA-5 (Ifih1) all lacked inverted SINEs, but harbored stretches rich in GT-repeats. We confirmed by qRT-PCR that each of these 3′UTRs were also specifically pulled down by the Z22 antibody in the absence of ADAR1, and that IFN treatment increased the abundance of these RNAs in Z22 pulldowns (see, FIG. 1, Panel l). Examining the GT-rich sequences of these non-SINE containing 3′UTRs indicate that they are potentially capable of folding into the shape of a dumbbell, with multiple putative core Za binding sites of 4 base pairs within a longer dsRNA duplex (see, FIG. 1, Panel l). To test if such a dumbbell sequence was prone to forming Z-RNA, we synthesized a consensus dumbbell comprising the potential Z-forming and Za binding core features observed in the Z22-enriched 3′UTR sequences (see, FIG. 1, Panel m), and examined its capacity to form Z-RNA in vitro. We found that the dumbbell, but not a control RNA, was capable of forming Z-RNA in vitro, although Z-formation required high salt concentrations (see, FIG. 1, Panel n).

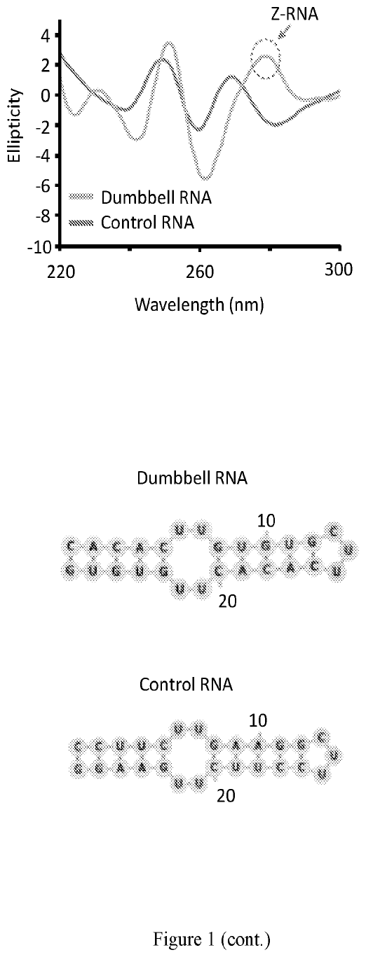

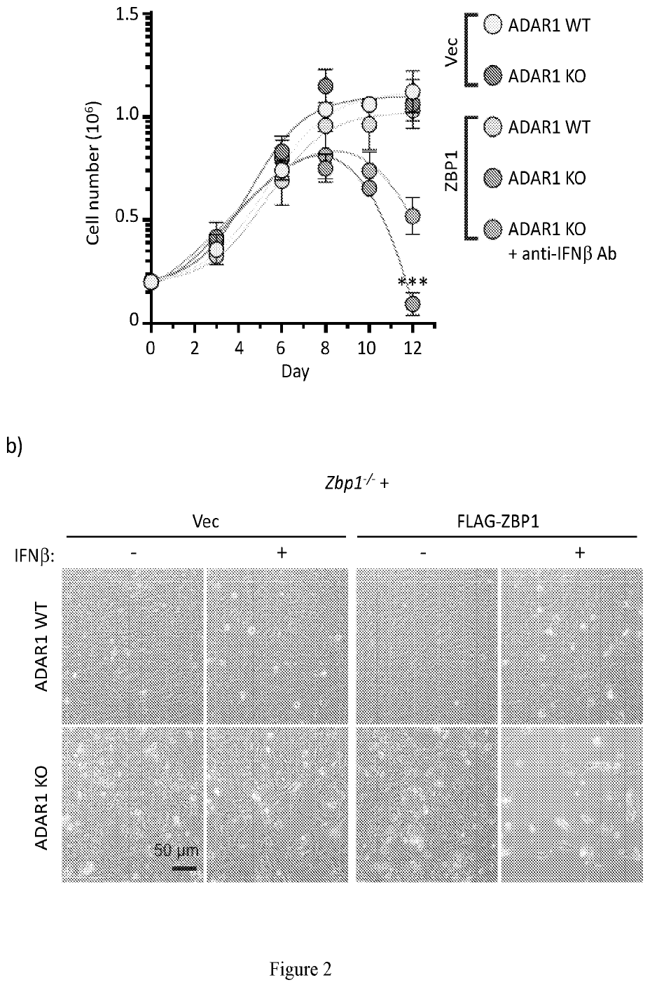

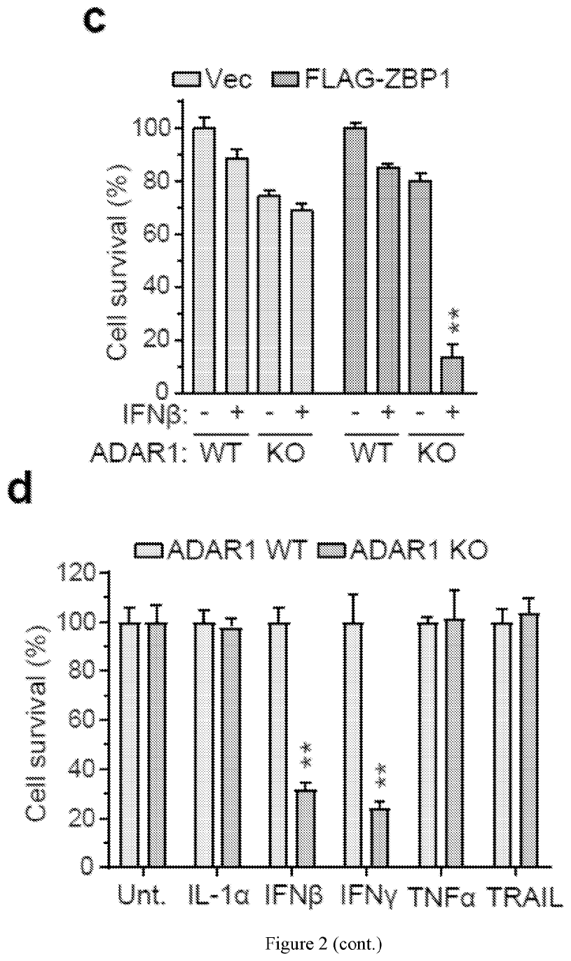

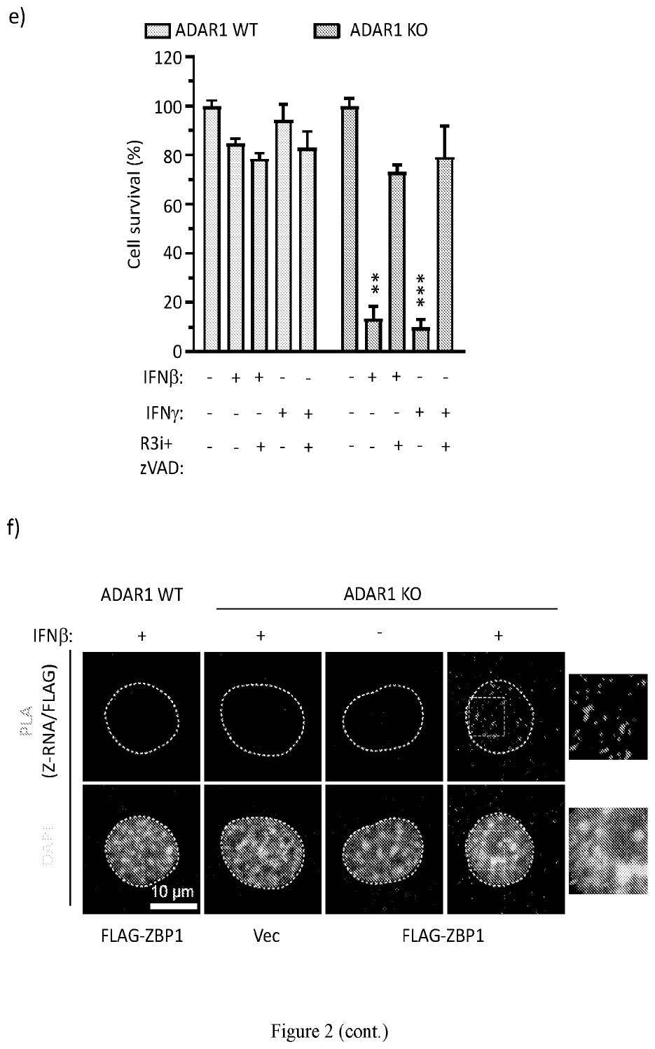

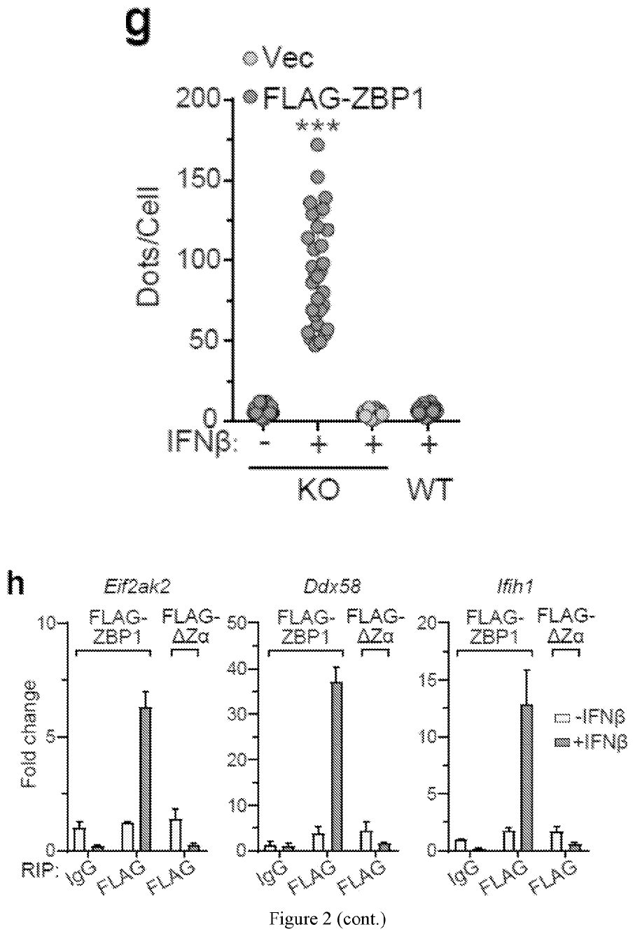

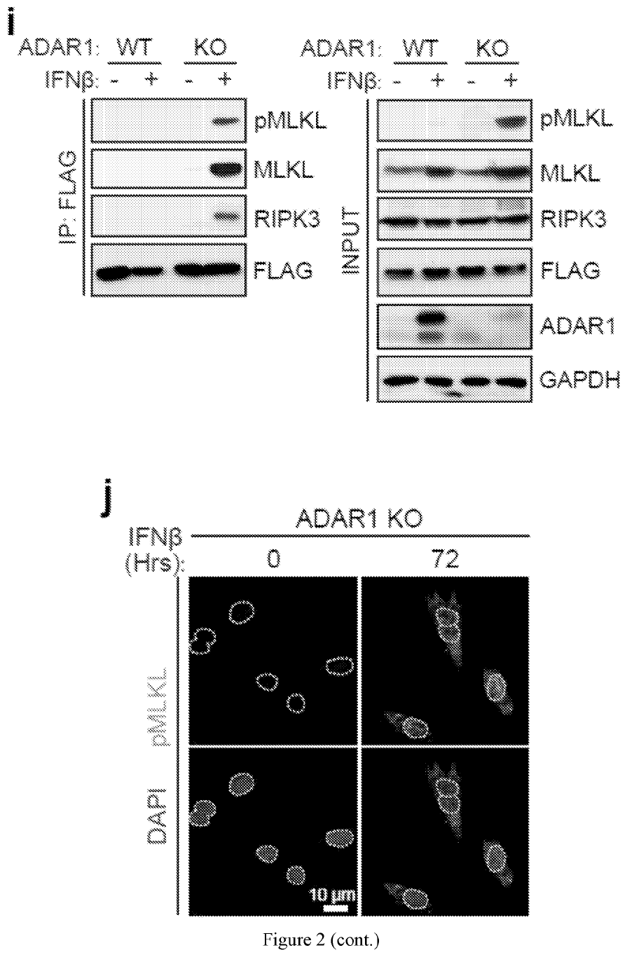

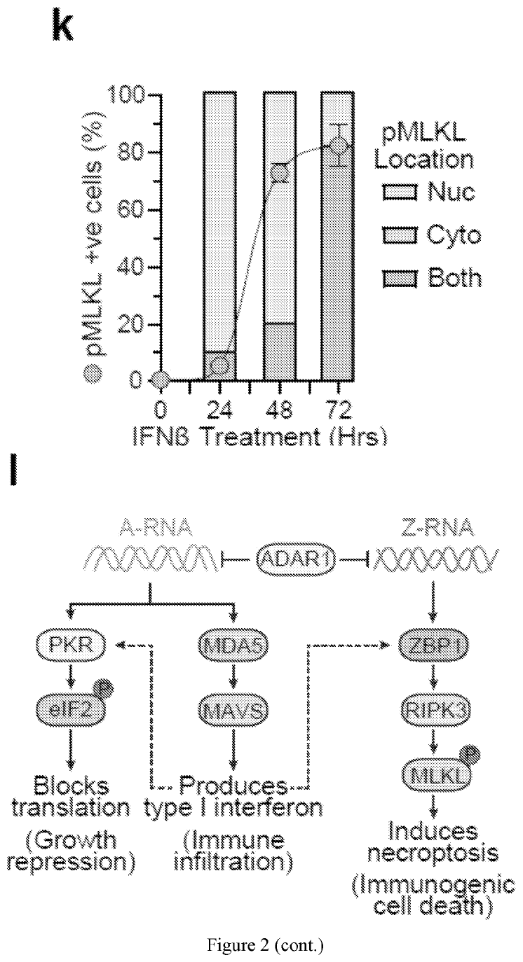

The high-salt requirement for Z-RNA formation of a synthetic Z-prone RNA in vitro is not unexpected, as additional determinants in the 3′UTRs of the endogenous RNAs may stabilize these dumbbell structures in the Z-conformation in cells. Moreover, binding to the Za domain can stabilize Z-prone RNAs in the Z-conformation, and as shown (see, FIG. 2, Panel h), the 3′UTRs of Eif2ak2, Ddx58 and Ifih1 were readily pulled down by FLAG-ZBP1 (but not a Zu mutant of ZBP1) from ADAR1 KO MEFs. Altogether, these results demonstrate that both inverted SINEs and SINE-independent GT-rich ‘foldback’ sequences within the 3′UTRs of mRNAs (particularly ISG mRNAs) form Z-RNAs in cells, and are substrates for ADAR1.

### Example 3: ADAR1 Loss Activates ZBP1-Driven Necroptosis

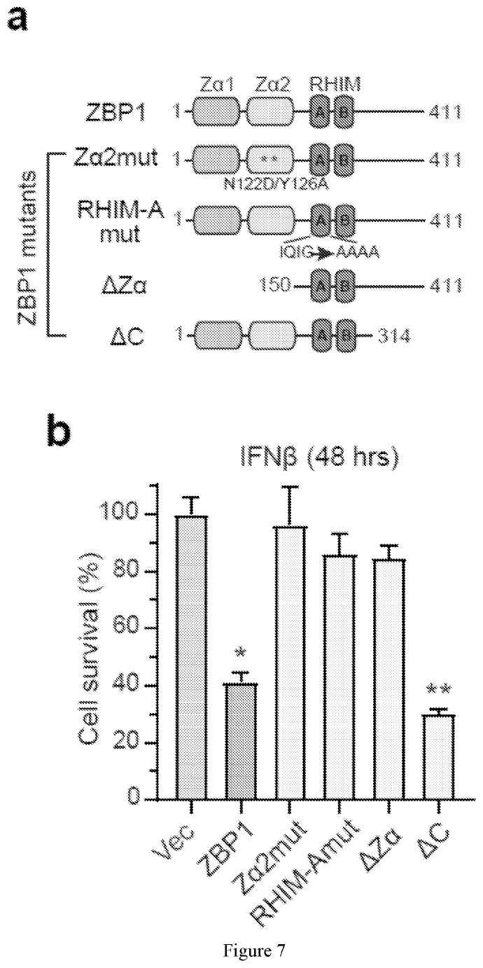

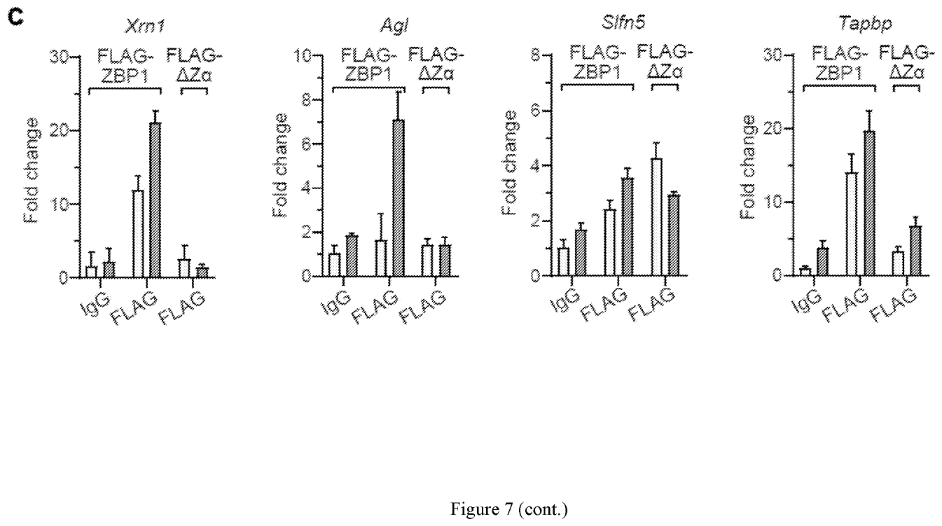

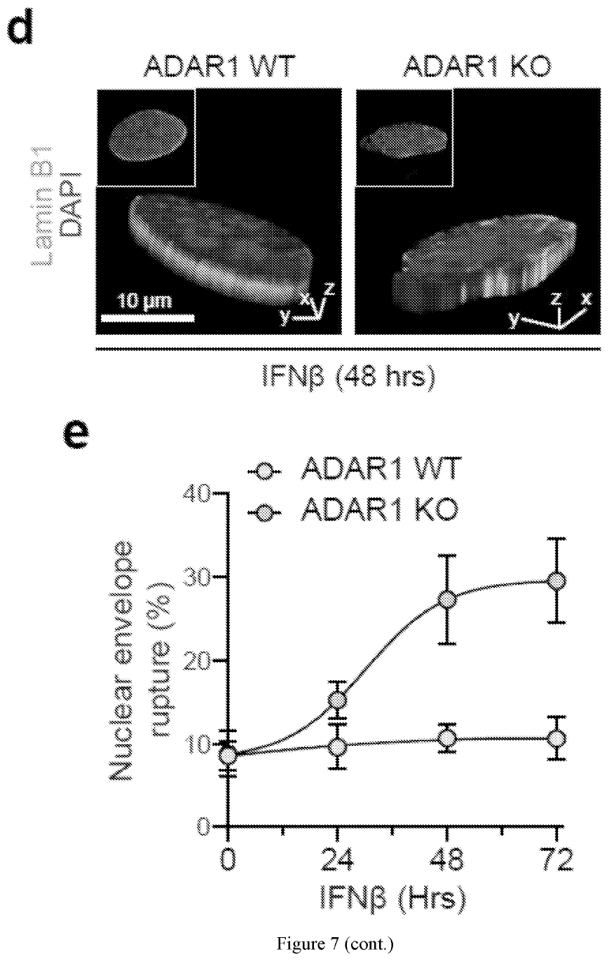

We noticed that MEFs in which ADAR1 was acutely ablated by CRISPR/Cas9 approaches began dying by 7 days post-ADAR1 loss, about 3 days after Z-RNA accumulation was first observed (see, FIG. 6, Panel a), suggesting that ADAR1 loss induces Z-RNA-triggered ZBP1-dependent cell death. To explore this possibility, we generated immortalized Zbp1^{-/-} MEFs stably expressing either FLAG-tagged wild-type murine ZBP1 or an empty vector control, ablated ADAR1 in these cells, and observed them over a 12-day period. ADAR1 loss in FLAG-ZBP1-reconstituted MEFs resulted in growth arrest by day 6, followed by progressive IFN-dependent loss of viability between days 7 and 12. In contrast, Zbp1^{-/-} MEFs carrying an empty vector underwent growth arrest by day 7, but remained viable for the duration of the study (see, FIG. 2, Panel a). Exposing ADAR1 KO cells to recombinant IFNβ triggered ZBP1-dependent cell death within 48 hours of treatment (see, FIG. 2, Panel b and Panel c). Of a panel of inflammatory cytokines tested, only IFNβ and IFNγ, but not IL-1α, TNFα, or TRAIL, induced ZBP1-dependent cell death in ADAR1-deficient MEFs (see, FIG. 2, Panel d). IFN-activated cell death was rescued by the combination of the pan-caspase blocker zVAD and the RIPK3 kinase inhibitor GSK'843 (R3i), demonstrating that death was a combination of apoptosis and necroptosis (see, FIG. 2, Panel e), as we have previously shown with IAV (Zhang et al., Cell, 2020, 180, 1115-1129 e1113). Cells reconstituted with a ZBP1 mutant lacking its Zα2 domain did not succumb to IFNβ upon ADAR1 ablation, suggesting that ZBP1 sensed endogenous Z-RNAs unleashed by ADAR1 loss to activate cell death (see, FIG. 7, Panel a). In agreement, we found that stimulating ADAR1 KO cells with IFNβ caused FLAG-ZBP1 to co-localized with nuclear Z-RNA (see, FIG. 2, Panel f and Panel g). Examined ZBP1-associated RNAs from IFNβ-treated ADAR1 KO cells by RT-qPCR showed that FLAG-ZBP1, (but not a Za deletion mutant [FLAG-ΔZα]), bound the 3′UTRs of all the Z-RNA-forming 3′UTRs identified in Z22 pulldowns (see, FIG. 2, Panel h and FIG. 7, Panel a, Panel b, and Panel c). Thus, Z-RNA sequences within the 3′UTRs of ISG mRNAs are bona fide ligands for ZBP1 activation in ADAR1-deficient cells (see, FIG. 2, Panel h). Following activation, ZBP1 complexed with RIPK3 and MLKL, and induced phosphorylation of MLKL in IFN-treated ADAR1 KO cells (see, FIG. 2, Panel i). In accordance with a role for the RIP Homology Interaction Motif (RHIM) in ZBP1 in nucleating RIPK3, a RHIM mutant of ZBP1 failed to induce cell death (see, FIG. 7, Panel d and Panel e). Phosphorylated MLKL was first seen in the nucleus, before migrating to the cytosol and the plasma membrane (see, FIG. 2, Panel j and Panel k). Notably, MLKL activation was associated with detectable rupture of the nuclear envelope in ˜30% of immortalized MEFs at 48 hours post IFNβ treatment (see, FIG. 7, Panel d). Rupture was first seen ˜24 hours post IFNβ exposure in ADAR1 KO FLAG-ZBP1 MEFs, shortly before loss of viability (see, FIG. 7, Panel e). Altogether, these findings show that endogenous Z-RNAs associate with and activate ZBP1 when ADAR1 is absent. ZBP1-driven necroptosis is thus a heretofore unappreciated arm of the innate immune response to endogenous dsRNA.

### Example 4: CBL0137 Induces Z-DNA Formation in Mammalian Genomic DNA

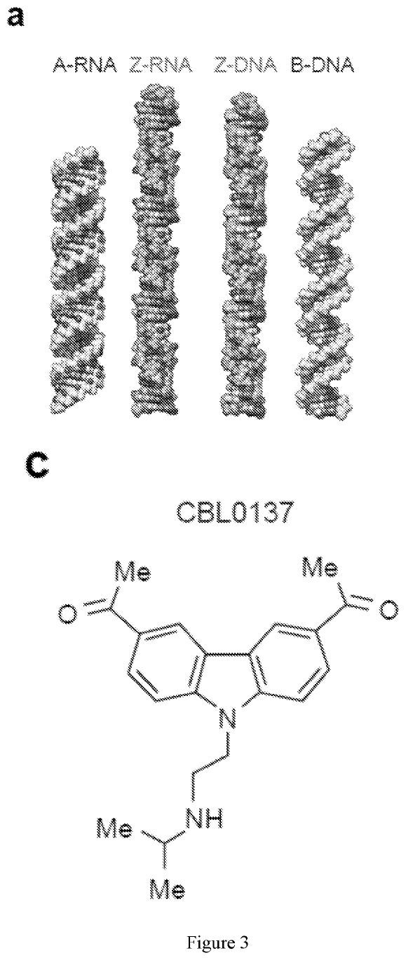

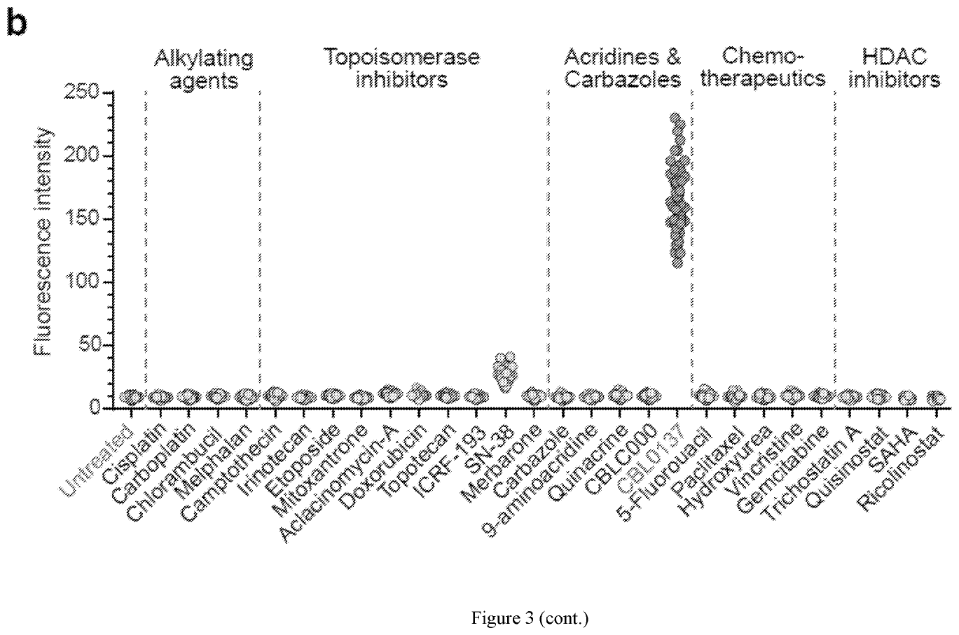

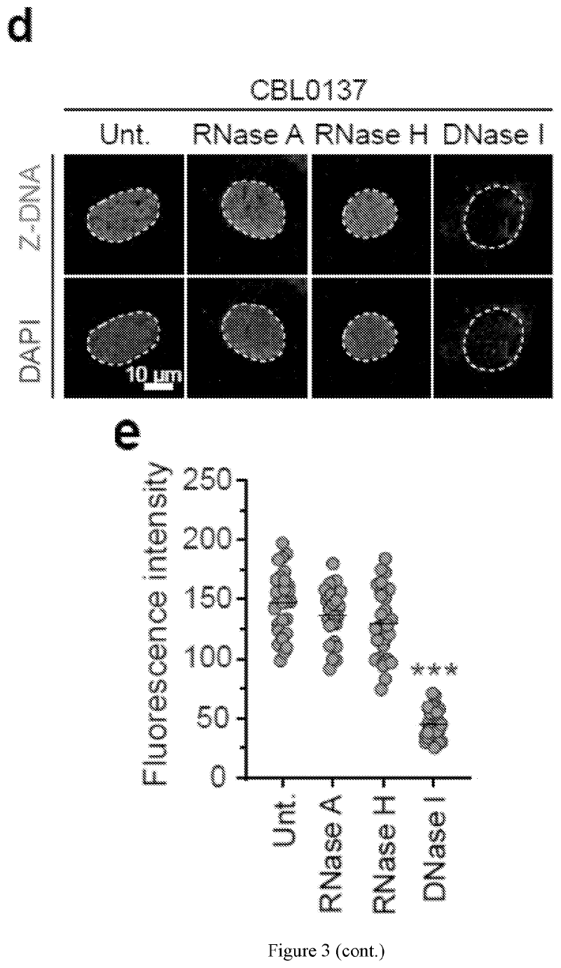

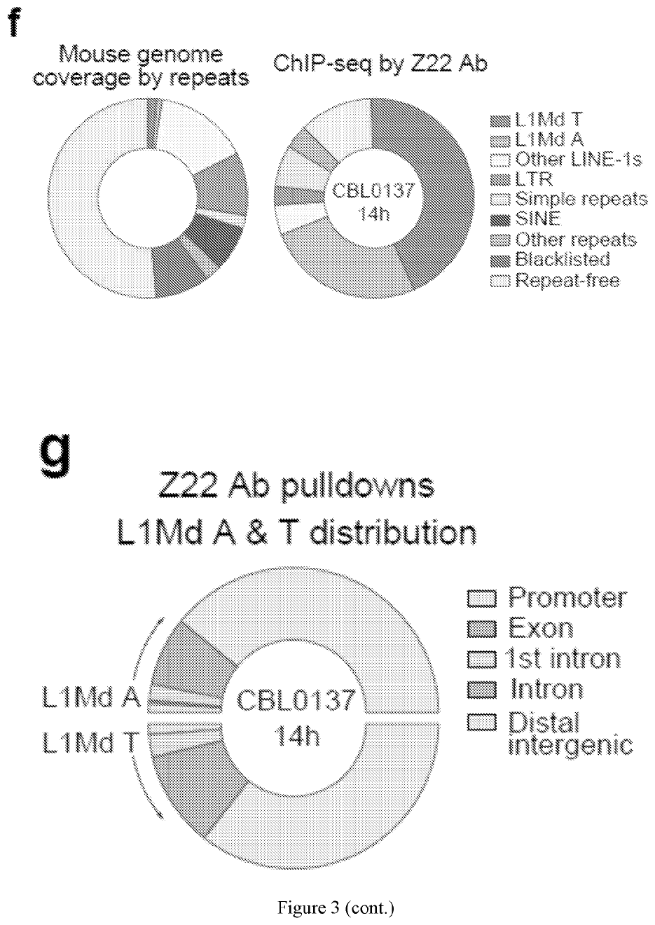

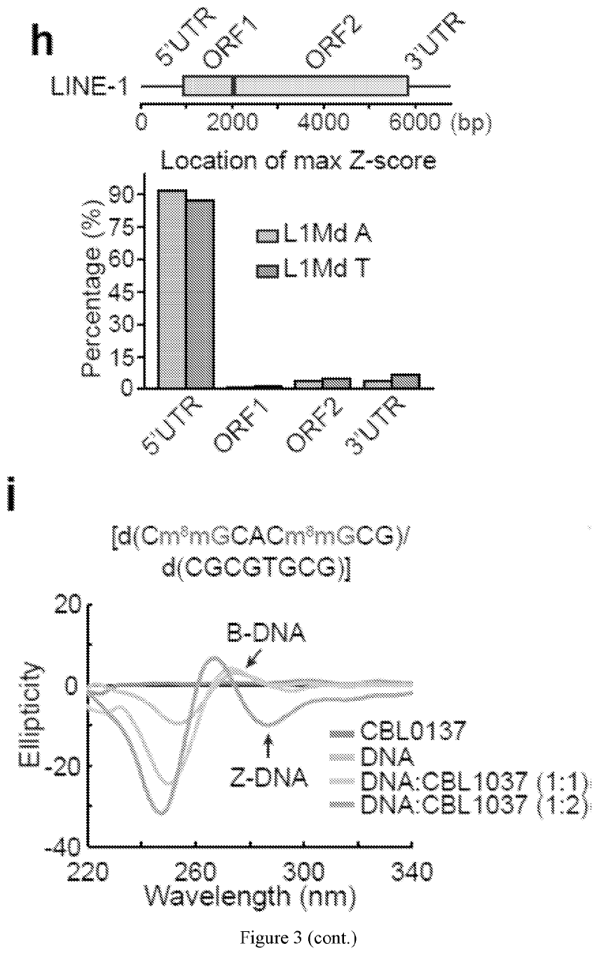

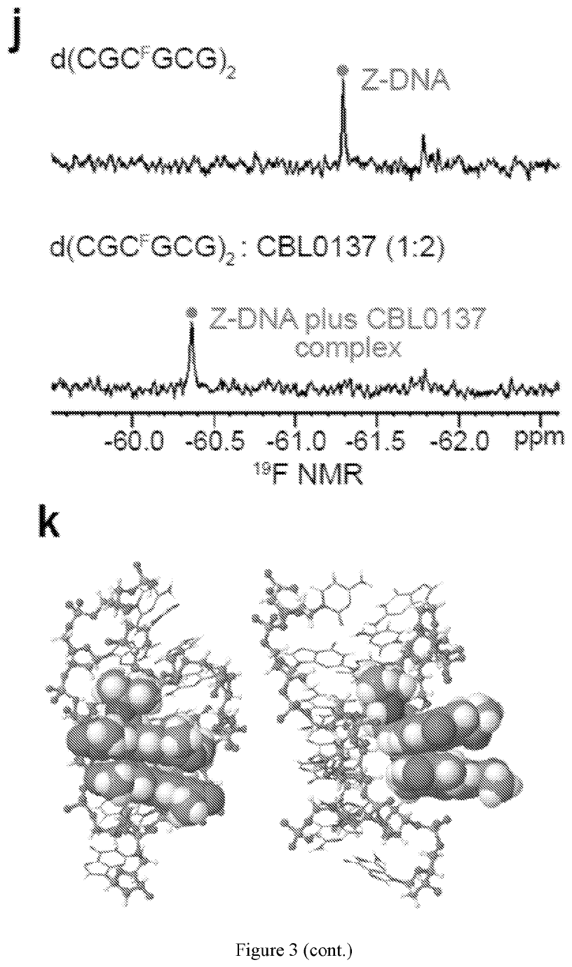

No clinically viable small-molecule strategies to block ADAR1 are currently available, and Z-RNA is difficult to produce by synthetic means (Hardin et al., Biochemistry, 1987, 26, 5191-5199; and Balasubramaniyam et al., Molecules, 2018, 23, 2572-2579). Z-RNA, however, shares almost-identical structures with Z-DNA (see, FIG. 3, Panel a). Like ADAR1, ZBP1 binds both Z-RNA and Z-DNA equally well in vitro (Brown et al., Proc Natl Acad. Sci. USA, 2000, 97, 13532-13536; and Kim et al., Proc. Natl. Acad. Sci. USA, 2011, 108, 6921-6926).

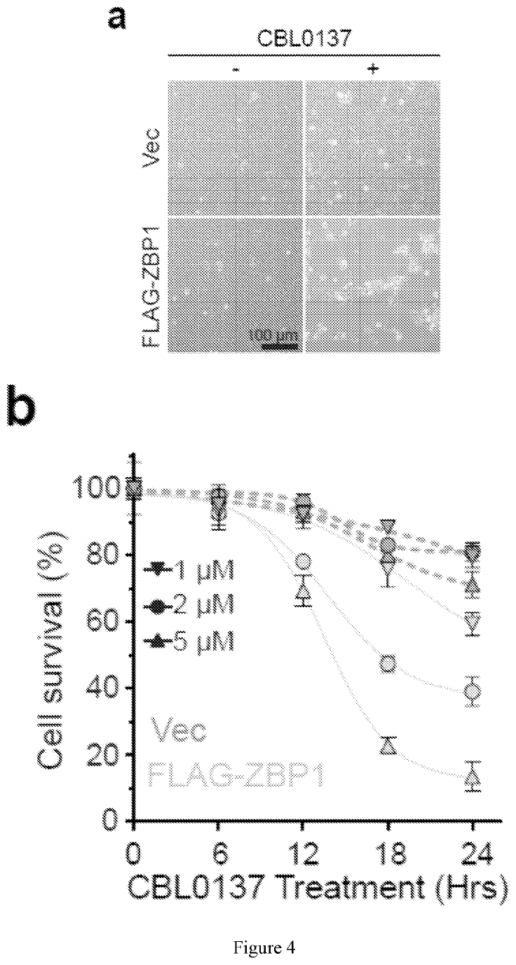

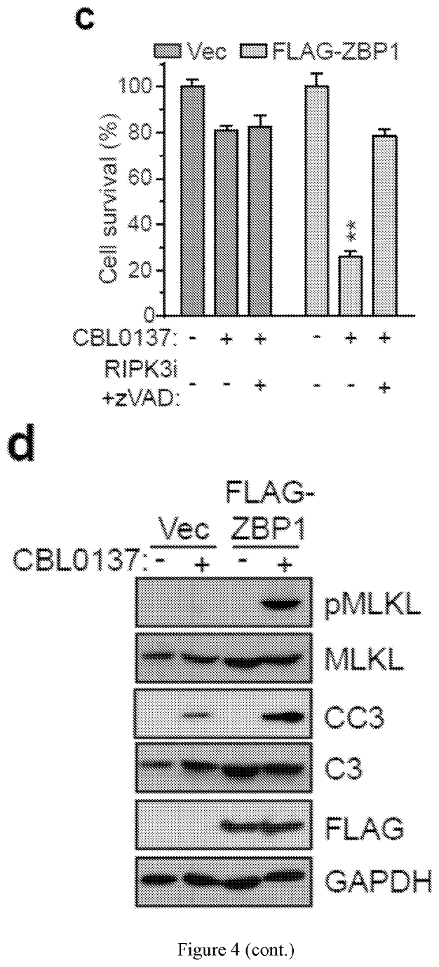

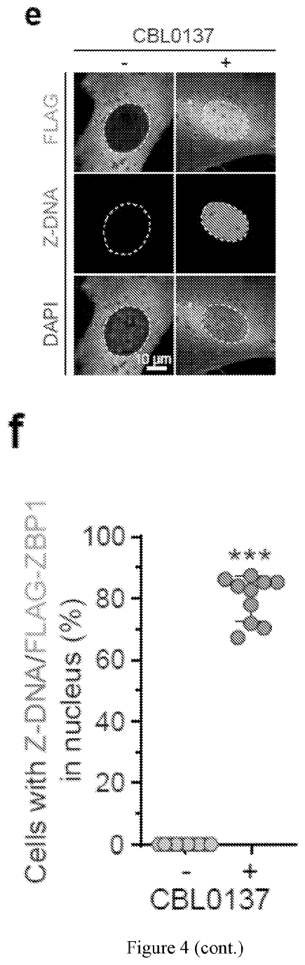

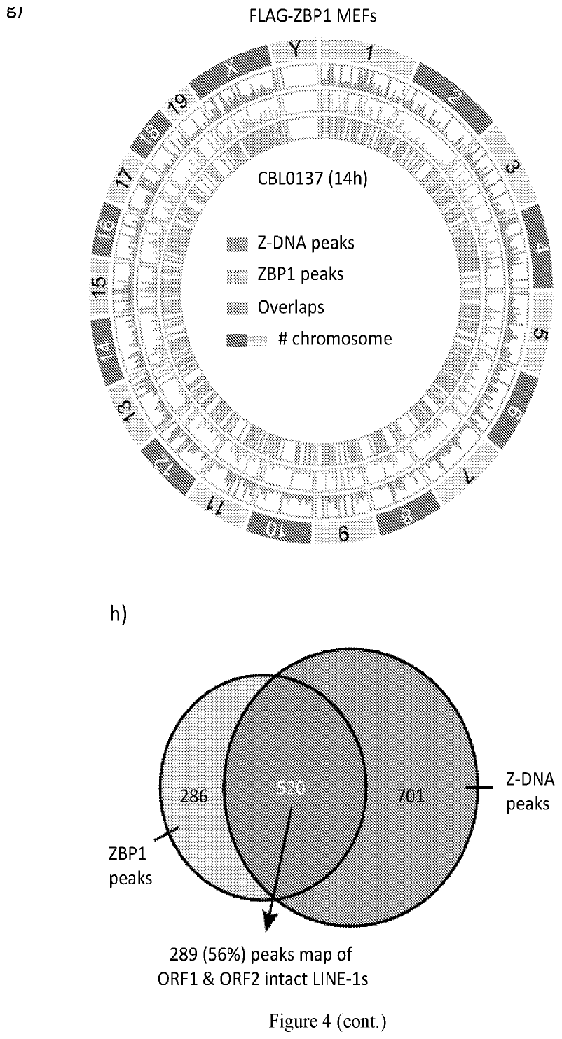

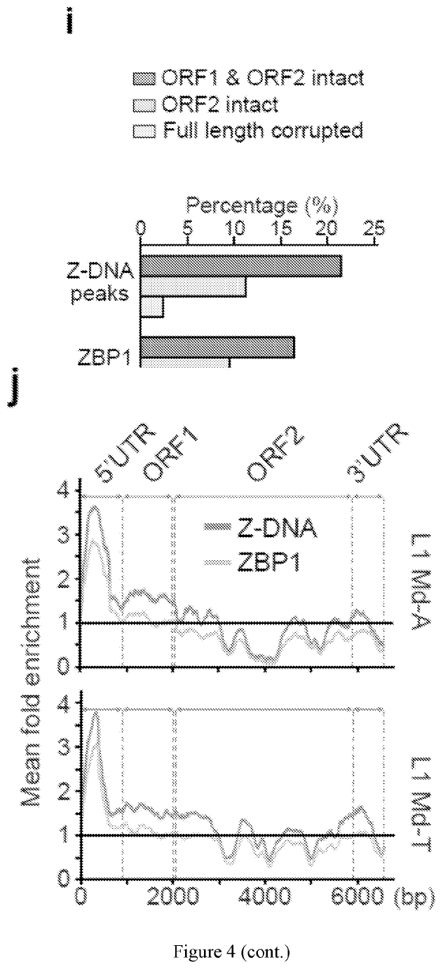

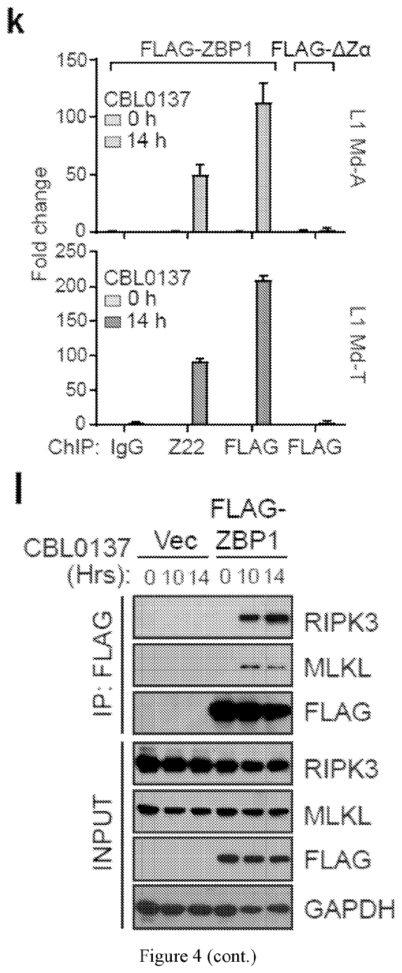

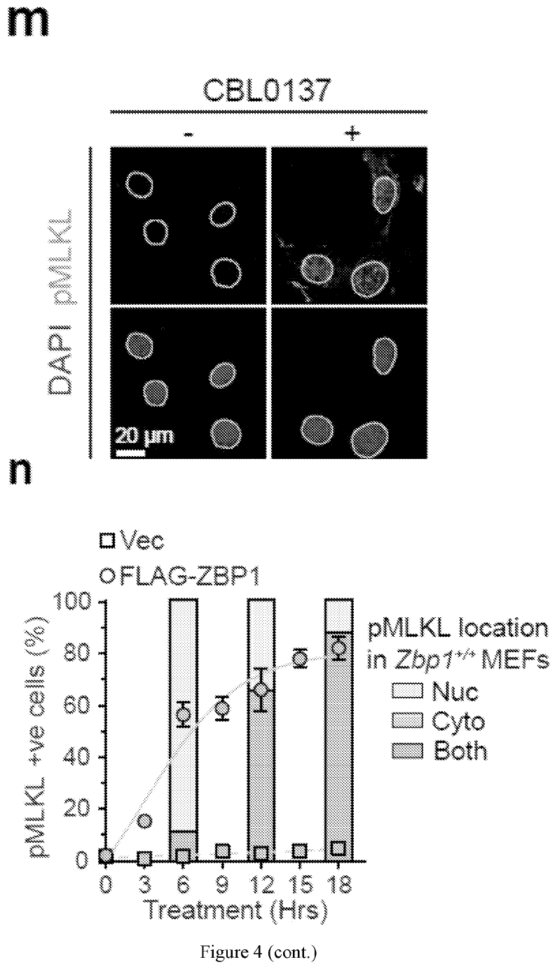

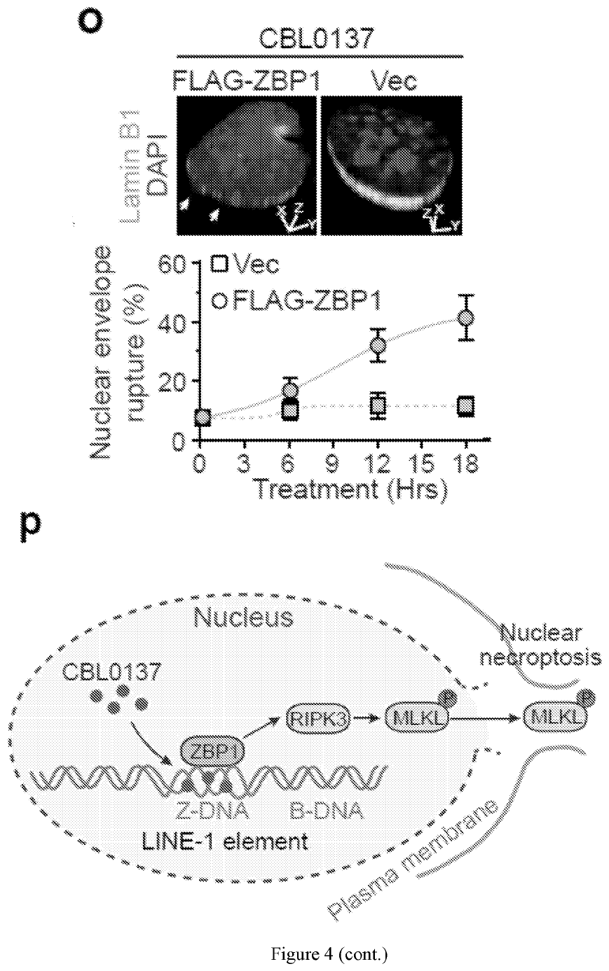

Importantly, Z-DNA can be generated in eukaryotic cells from negative supercoiling or epigenetic modification of genomic DNA (Haniford et al., Nature, 1983, 302, 632-634; Peck et al., Proc. Natl. Acad. Sci. USA, 1982, 79, 4560-4564; and Herbert et al., J. Biol. Chem., 1996, 271, 11595-11598). Reasoning that agents capable of generating Z-DNA in cells will directly activate ZBP1, overriding the need either for either synthetic Z-RNA (or Z-DNA) or ADAR1 inhibitors, we carried out a curated screen for small-molecule inducers of Z-DNA formation, focusing on compounds known to intercalate DNA, affect DNA topology, or alter chromatin structure and dynamics. From this screen, we identified the second-generation curaxin family member CBL0137 as a potent inducer of Z-DNA in mammalian cells (see, FIG. 3, Panel b and Panel c). CBL0137 is best-characterized as an inhibitor of the histone chaperone FACT (Chang et al., J Cancer Metastasis Treat, 2019, 5), but also directly associates with DNA (Safina et al., Nucleic Acids Res, 2017, 45, 1925-1945). By immuno-precipitating and sequencing Z-DNA from CBL0137-treated MEFs, we found that CBL0137 induced Z-DNA formation in hundreds of sites across the mouse genome, the majority of which mapped to long interspersed nuclear element LINE1 retroviral DNA. In particular, LINE1 elements encoding full-length L1Md A and L1Md T were highly enriched in Z22 pulldowns of CBL0137-treated cells (see, FIG. 3, Panel f, right) compared to their frequency within the mouse genome (see, FIG. 3, Panel f, left). L1Md A and L1Md T forming Z-DNA in CBL0137-treated cells were mainly intergenic, and rarely seen within exons (see, FIG. 3, Panel g). L1Md A and L1Md T make up less than 12% (see, FIG. 3, Panel f) of all repeat elements, but constitute around 59% of the full-length transposons in mouse (Sookdeo et al., Mob DNA, 2013, 4, 3). In examining the sequences of these two L1 elements, we noticed that their 5′UTRs contained GC-rich sequences and are predicted by our DeepZ algorithm (Beknazarov et al., Sci Rep, 2020, 10, 19134) to be predisposed to Z-DNA formation (see, FIG. 3, Panel h). As shown (see, FIG. 4, Panel) the Z22 binding peak was also within the 5′UTRs of these L1 elements, situated ˜450 bp from their 5′ termini, suggesting that CBL0137 may trigger Z-DNA formation by inducing the B→Z conversion of Z-prone B-DNA. To directly test if CBL0137 could convert Z-prone B-DNA to Z-DNA in vitro, we synthesized a GC-rich B-DNA octamer incorporating 2′-O-methyl-8-methyl modification of internal guanosine nucleosides (m^{g}Gm) in one strand of the duplex. The introduction of a methyl group at the C8 position strongly facilitates the B→Z transition (Bao et al., Curr Protoc, 2021, 1, e28). In the absence of CBL0137, the m⁸Gm dsDNA octamer was in the B-conformation when examined by circular dichroism, but flipped into the Z-conformation with the addition of equimolar (1:1) amounts of CBL0137 (see, FIG. 3, Panel i). This effect was even more pronounced at a higher ratio (2:1) of CBL0137 to B-DNA (see, FIG. 3, Panel i). The formation of a CBL0137:Z-DNA complex was confirmed by ¹⁹F NMR (see, FIG. 3, Panel j). In agreement with these observations, Molecular Dynamic Stimulation of the putative CBL0137:Z-DNA complex showed that two stacked CBL0137 molecules can be accommodated in the minor groove of Z-DNA (see, FIG. 3, Panel k), suggesting that CBL0137 can ‘lock’ dsDNA in the Z-conformation, following the B→Z transition.

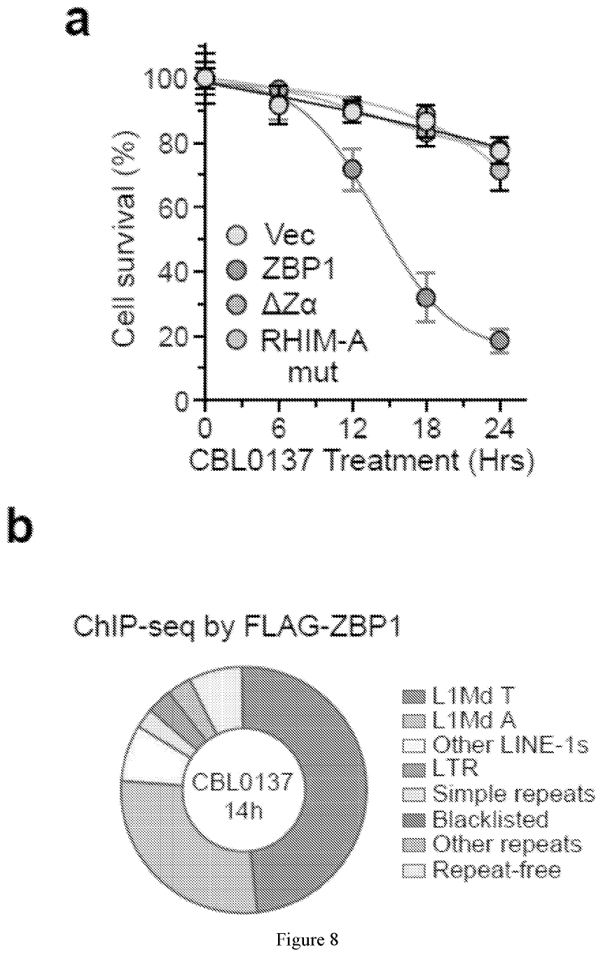

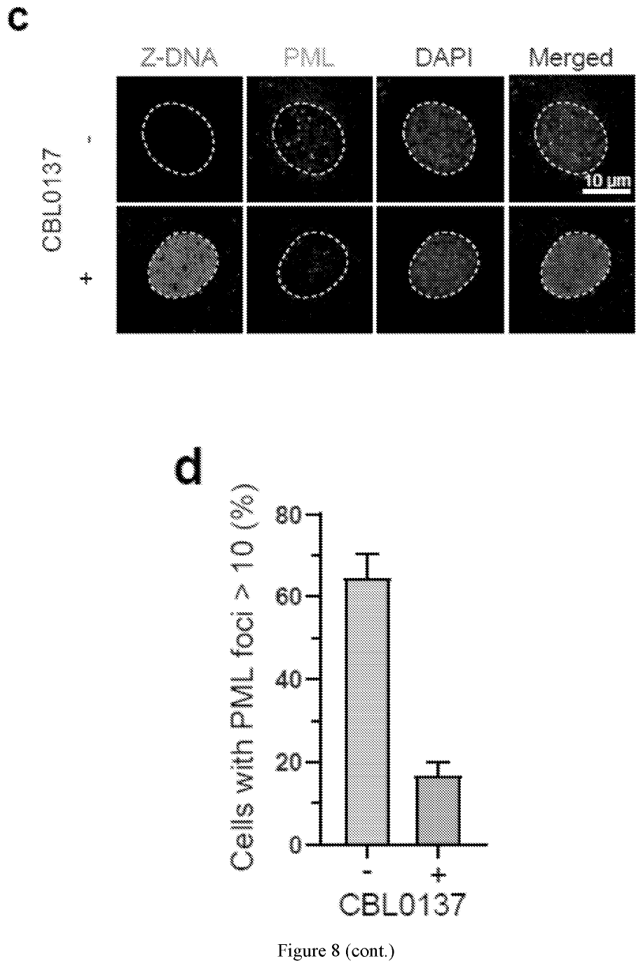

In addition to localizing to Z-prone B-DNA sequences and flipping these into Z-DNA, CBL0137 might also induce a B→Z transition in cells by displacing FACT or other proteins from histone linker regions, either directly or by intercalation into B-DNA, resulting in topological stress and Z-DNA formation. This possibility is supported by our finding that DNA sequences in Z22 pulldowns were also enriched in epigenetic marks (H2A.Z) associated with FACT activity (Jeronimo et al., Mol Cell, 2015, 58, 1113-1123). Interestingly, CBL0137 induced the loss of PML bodies normally associated with suppression of L1 elements (Denli et al., Cell, 2015, 163, 583-593) (see, FIG. 8, Panel c), suggesting that Z-DNA formation may also arise from Li transcription; these possibilities warrant further exploration.

### Example 5: CBL0137 Potently Triggers ZBP1-Dependent Necroptosis

We found that CBL0137 at 5 μM induced robust ZBP1-dependent death in ˜90% of MEFs by 24 hours (see, FIG. 4, Panel a and Panel b). CBL0137 induced cell death required the Zai domains and RHIM of ZBP1 (see, FIG. 8, Panel a), and was blocked by the combination of zVAD and the RIPK3 inhibitor GSK'872 (see, FIG. 4, Panel c), demonstrating that it was a combination of apoptosis and necroptosis, similar to what we have observed in other scenarios of ZBP1 activation, such as upon ADAR1 loss (see, FIG. 2, Panel e) and following IAV infection (Zhang et al., Cell, 2020, 180, 1115-1129 e1113). ZBP1-induced cell death resulted in the phosphorylation of MLKL and the cleavage of caspase-3 (see, FIG. 4, Panel d). CBL0137 also induced ZBP1-independent caspase activity (see, FIG. 4, Panel d) in MEFs, which manifested as apoptosis between 18 hours and 24 hours post treatment, likely accounting for its oncocidal effects in tumor derived-cell lines, most of which are RIPK3-deficient (Somers et al., Int J Cancer, 2020, 146, 1902-1916; Gasparian et al., Science translational medicine, 2011, 3, 95ra74; Carter et al., Science translational medicine, 2015, 7, 312ra176; and Koo et al., Cell Res, 2015, 25, 707-725). CBL0137 triggered the translocation of ZBP1 into the nucleus (see, FIG. 4, Panel e) and its co-localization with Z-DNA in most treated cells (see, FIG. 4, Panel f).

We next treated immortalized Zbp1^{-/-} MEFs expressing FLAG-ZBP1 with CBL0137 for 14 hours, immunoprecipitated FLAG-ZBP1 from these cells, and performed ChIP-Seq on eluted DNA. ZBP1-bound DNAs mapped to numerous sites across the entire mouse genome (see, FIG. 4, Panel g, blue peaks—might not be necessary). A large fraction of fragments bound by ZBP1 (see, FIG. 4, Panel g, purple bars—might not be necessary) overlapped with those sites detected by ChIP-seq with the Z-DNA specific Z22 antibody after CBL0137 exposure (see, FIG. 4, Panel g, magenta peaks—might not be necessary). The majority (58%) of overlapping peaks bound by both Z22 and ZBP1 were full-length LINE1 elements (see, FIG. 4, Panel h and Panel i). As with Z22 (see, FIG. 3, Panel h), most of these ZBP1-bound sequences mapped to the 5′UTRs of Li Md A and Md T elements (see, FIG. 4, Panel j and FIG. 8, Panel b), confirming that these sequences were prone to forming Z-DNA. We validated by RT-qPCR that both the Z22 antibody and FLAG-ZBP1, but not an isotype control antibody (IgG) or FLAG-ΔZα, bound the 5′UTRs of L1 Md A and Md T elements following CBL0137 treatment (see, FIG. 3, Panel k). Consequently, CBL0137 triggered an association between ZBP1, RIPK3 and MLKL (see, FIG. 4, Panel i), causing the activation of MLKL that was first observed in the nucleus (see, FIG. 4, Panel m), and then in the cytoplasm (see, FIG. 4, Panel n). ZBP1-dependent rupture of the nuclear envelope (see, FIG. 4, Panel o) resulting in nuclear necroptosis (see, FIG. 4, Panel p).

### Example 6: CBL0137 Reverses ICB Resistance by Inducing ZBP1-Initiated Necroptosis in Fibroblasts of the TME

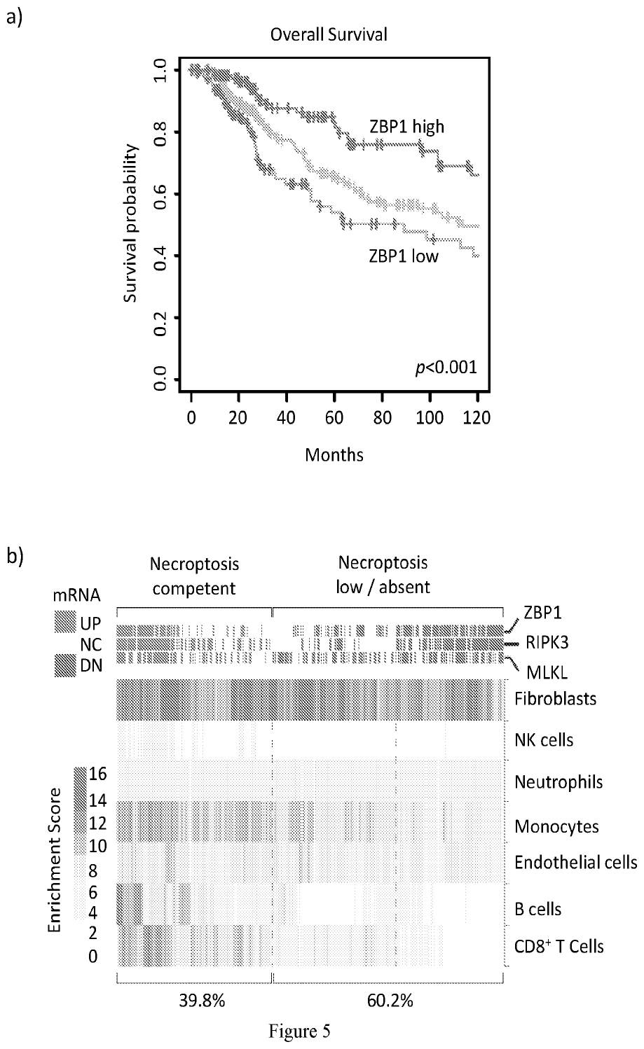

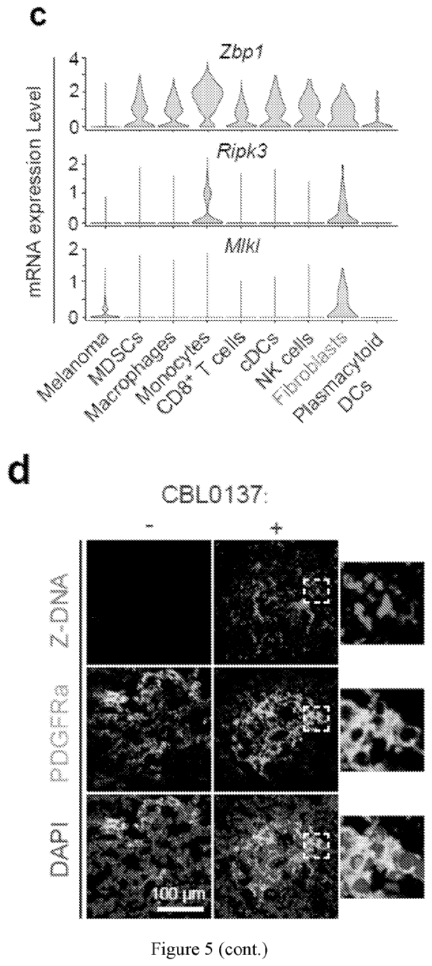

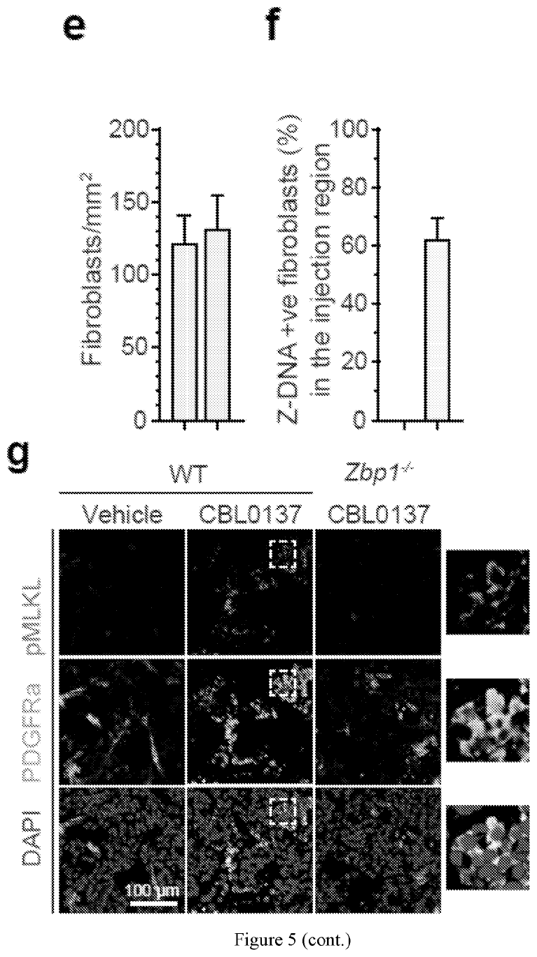

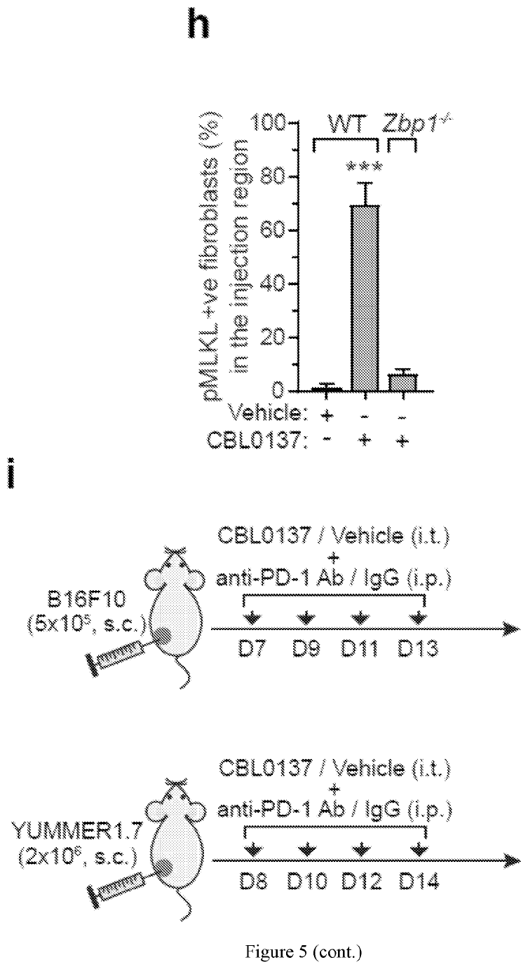

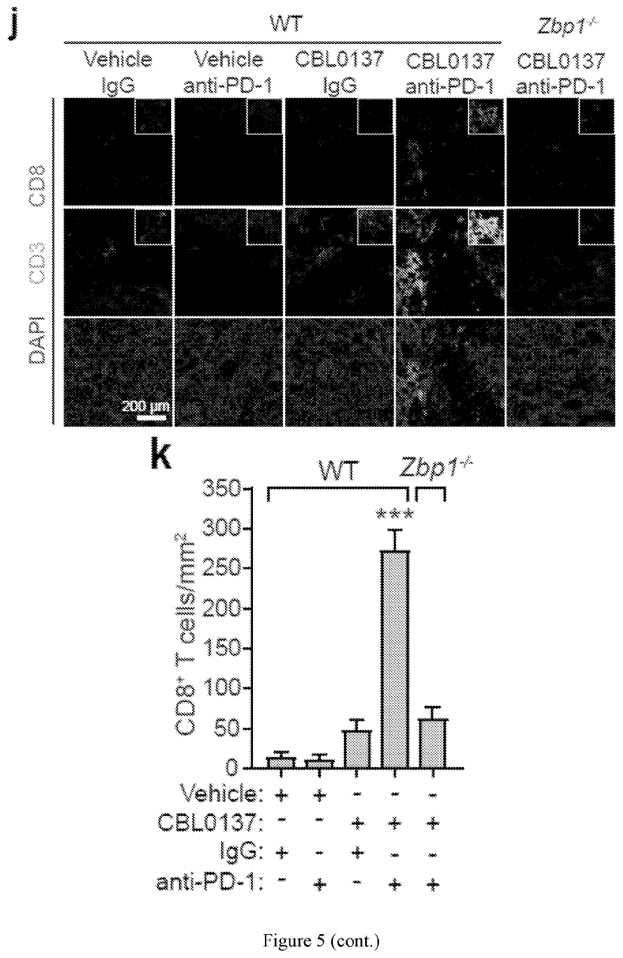

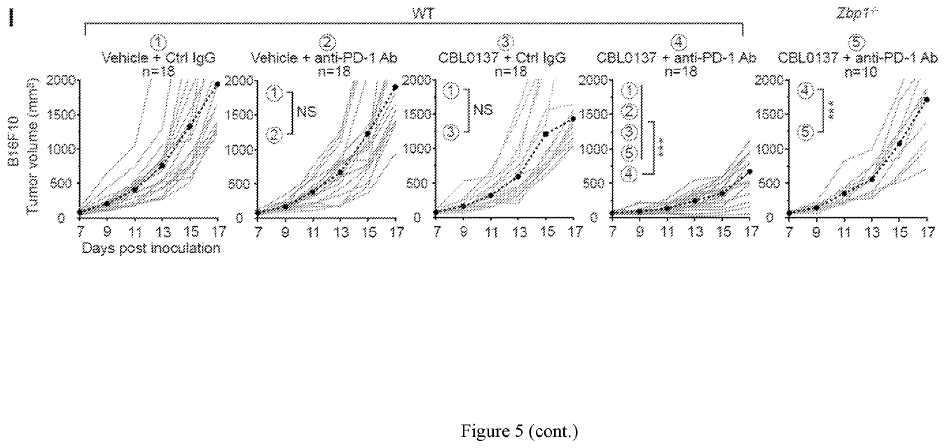

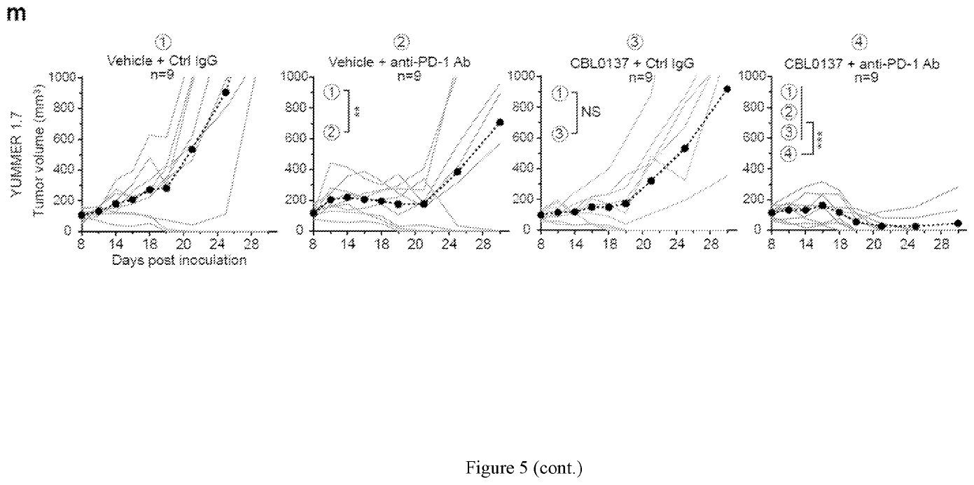

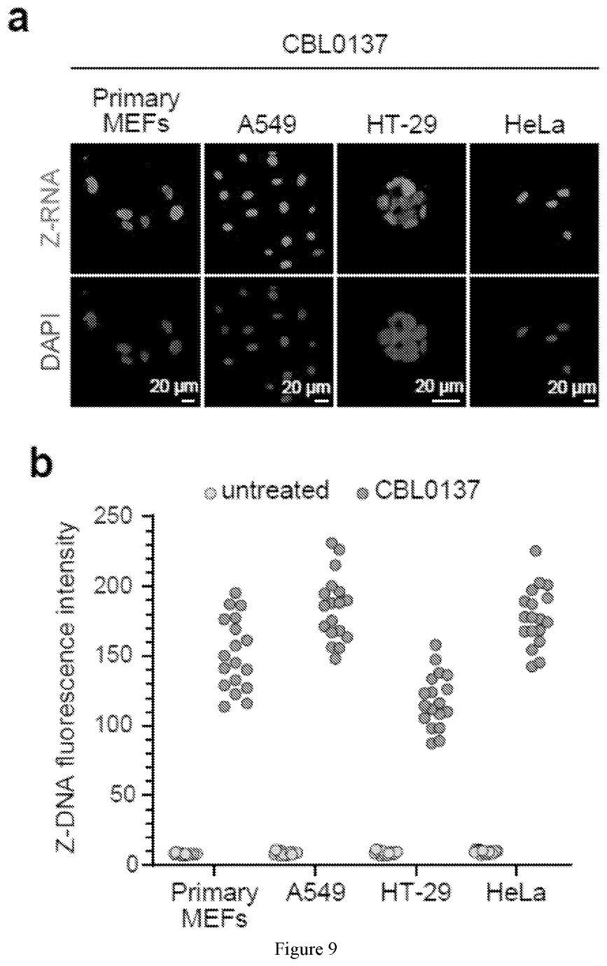

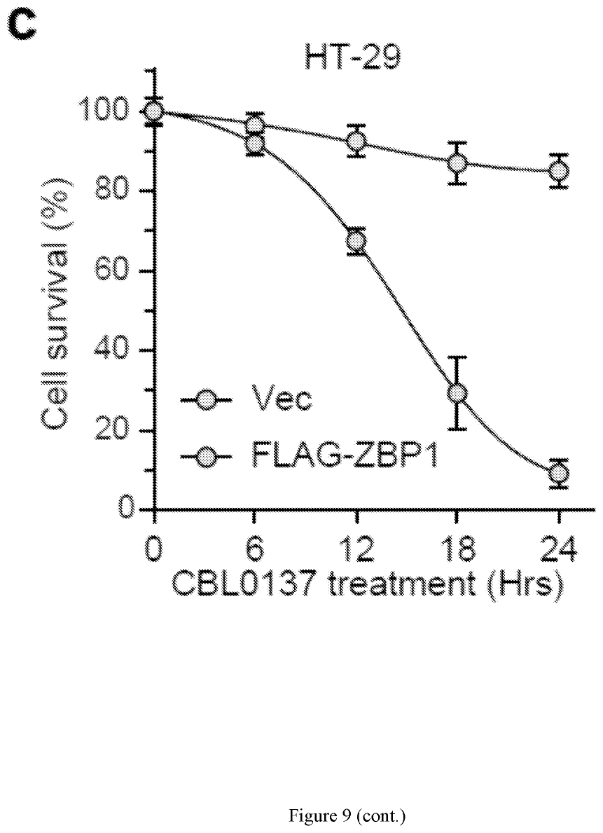

CBL0137 induced Z-DNA formation in all mammalian cell lines tested, including in human lines (see, FIG. 9, Panel a and Panel b), and initiated robust ZBP1-dependent cell death in human cells (see, FIG. 9, Panel c), suggesting that this compound will be useful as a potential ZBP1 agonist in multiple human cancers. To leverage our findings for cancer immunotherapy, we first carried out an analysis of solid tumors in The Cancer Genome Atlas (TCGA), and found that Zbp1 expression levels were highly correlated with better survival outcomes in malignant melanoma, a tumor type in which ICB has shown promise, but where therapy resistance is a significant problem (ref 50,51) (see, FIG. 5, Panel a). Tumors with an intact necroptosis machinery showed significantly higher levels of infiltrating monocytes, cDCs, and CD8⁺ T cells, indicating that activation of ZBP1-dependent necroptosis in tumors has the potential to trigger beneficial adaptive immune responses in melanoma (see, FIG. 5, Panel b). Unfortunately, a majority (˜60%) of tumors were either low in necroptosis gene expression, or did not express the necroptosis machinery to any detectable extent, paralleling what is seen in most human tumor-derived cell lines (Koo et al., Cell Res, 2015, 25, 707-725; and He et al., Cell, 2009, 137, 1100-1111). These tumors were also proportionately devoid of immune cells (see, FIG. 3, Panel b). Nonetheless, all melanomas showed significant fibroblastic infiltrate (see, FIG. 3, Panel b), and an analysis of scRNA-Seq data from the ICB-refractory murine melanoma (B16-F10) showed that tumor infiltrating fibroblasts express the necroptosis machinery (see, FIG. 5, Panel c). These observations allowed us to hypothesize that inducing necroptosis by triggering ZBP1 activation in TME stromal cells (such as cancer-associated fibroblasts) would induce potent antitumor responses and potentiate ICB therapy. Further, such a novel approach would be of great clinical significance as it does not require that cancer cells be necroptosis-competent. To test this hypothesis, we generated B16-F10 melanoma xenografts on wild-type C57Bl/6 mice, and injected CBL0137 into these xenografts. We then assessed Z-DNA formation 16 hours and necroptosis activation 24 hours post-injection. We found that CBL0137 induced rampant Z-DNA formation in cells surrounding the injection site, including in fibroblasts of the TME, as determined by co-staining tumor sections for Z-DNA and the fibroblast marker PDGFRa (see, FIG. 5, Panel d). Quantifying these results showed that CBL0137 induced Z-DNA formation in >60% of TME fibroblasts, without altering the numbers of these cells (see, FIG. 5, Panel e). Staining sections from CBL0137-treated B16-F10 tumors generated in wild-type or Zbp1^{-/-} mice for pMLKL showed that CBL0137 activated necroptosis in a majority (˜70%) of TME fibroblasts. Importantly, necroptosis was dependent on host expression of ZBP1.

To examine whether necroptosis induced by CBL0137 in TME fibroblasts was able to reverse ICB non-responsiveness in a mouse model, we produced B16-F10 xenograft tumors in either wild-type or Zbp1^{-/-} mice. We then treated these mice with four cycles of CBL0137 or a vehicle control (intralesionally), in combination with either an anti-PD-1 antibody or an isotype control antibody (systemically, by intraperitoneal administration) (see, FIG. 5, Panel i). We found that the combination of CBL0137 and anti-PD-1 antibody, but not either agent alone, induced recruitment of CD3⁺CD8⁺ T cells into the tumor by D11 post-initiation of treatment (see, FIG. 3, Panel j and Panel k). T cell influx into the tumor was only seen in wild-type animals, and not in Zbp1^{-/-} mice (see, FIG. 3, Panel j and Panel k), demonstrating that ZBP1-dependent necroptosis was responsible for their recruitment. In agreement with these observations, anti-PD-1 antibody, when combined with CBL0137 was able to induce significant regression of B16-F10 tumors, but only in wild-type mice, and not in Zbp1^{-/-} mice (see, FIG. 5, Panel i). We next tested the effects of CBL0137 on anti-PD-1 antitumor responses on YUMM 1.7 xenografts. This model is considered more clinically-relevant than the B16-F10 model because YUMM 1.7 cells were obtained from a UV-induced murine melanoma, and because they are partially responsive to anti-PD-1 monotherapy (Wang et al., Pigment Cell Melanoma Res, 2017, 30, 428-435) mirroring human melanoma (Weiss et al., Clin Cancer Res, 2019, 25, 5191-5201; and Ribas et al., Science, 2018, 359, 1350-1355). In these xenografts, we found that anti-PD-1 antibody treatment by itself was able to induce the regression of a significant fraction (4/9) of tumors (see, FIG. 5, Panel m). While CBL0137 on its own resulted in slower tumor growth, compared to vehicle controls, the combination of CBL0137 and anti-PD-1 antibody induced complete or near-complete tumor regression in all (9/9) treated mice. Altogether, these encouraging results demonstrate that CBL0137, by inducing ZBP1-dependent necroptosis in cells of the TME, drives CD8⁺ T cell recruitment into tumors and strongly potentiates ICB responses in vivo.

### Example 7: Discussion

ADAR1 has been shown to negatively regulate type I interferon responses by editing A-RNAs in EREs and repressing further activation of MDA-5 and PKR. We now demonstrate that ADAR1 also represses Z-RNA formation and ZBP1-dependent necroptosis by preventing the accumulation mRNA transcripts that form Z-RNAs. Such Z-forming sequences are enriched in the 3′ UTRs of ISG mRNAs harboring either inverted SINEs or GT-rich segments capable of folding into Z-prone dumbbells. This work extends the range of sequences (which we call ‘flipons’ (Herbert et al., R Soc Open Sci, 2020, 7, 200222) known to form Z-DNA and Z-RNA in cells.

Currently no clinically-viable ADAR1 inhibitors or ZBP1 agonists exist. We report here a small molecule approach that bypasses the need for ADAR1 inhibition and activates ZBP1 directly by forcing endogenous flipons to form Z-DNA. By doing so, we induce a robust anti-tumor response that overcomes ICB non-responsiveness. This strategy employs the drug CBL0137 to drive Z-DNA formation in vivo. DNA weaponized in this manner bypasses ADAR1 dependent immune silencing within tumor cells to activate ZBP1 dependent ‘nuclear necroptosis’ in tumor stromal fibroblasts. We have previously shown (Zhang et al., Cell, 2020, 180, 1115-1129 e1113) that such nuclear necroptosis is significantly more immunogenic than conventional (i.e., cytoplasm-induced) necroptosis as it results in the release of numerous DAMPs (such as HMGB-1, IL-33, and, indeed, DNA itself), from the nucleus. CBL0137 was able to reverse ICB unresponsiveness in a refractory model of malignant melanoma, as well as in a clinically-relevant model of this malignancy. Both results demonstrate the potential for synergy of CBL0137 with ICB therapies. Importantly, CBL0137 manifested these effects by activating ZBP1 in fibroblasts of the TME, rather than in the tumor cells themselves, indicating that it will have therapeutic benefit even when cancer cells are necroptosis-incompetent. It is likely that previous studies examining ADAR1 loss (Ishizuka et al., Nature, 2019, 565, 43-48; and Chung et al., Cell, 2018, 172, 811-824 e814) failed to uncover ZBP1-driven necroptosis as a component of the immunogenic arsenal uleashed by activation of EREs, because many tumor-derived cell lines, including the ones used in those studies, do not express RIPK3 (Geserick et al., Cell Death Dis, 2015, 6, e1884; and Morgan et al., BMB reports, 2015, 48, 303-312).

Notably, CBL0137 has been used in Phase 1b clinical trials in humans, without significant systemic toxicity, perhaps because of low/absent basal ZBP1 levels in many normal cell types (ref). ZBP1 expression is, however, strongly induced by IFN, and the TME of many tumors often display a chronic IFN signature (Nirschl et al., Cell, 2017, 170, 127-141 e115; and Benci et al., Cell, 2016, 167, 1540-1554 e1512). Tumor-specific production of IFN may induce ZBP1 expression selectively in cells of the TME, offering an unexpected therapeutic window for both intralesional and systemic administration of CBL0137 in clinical settings. As the genes encoding MLKL, MDA-5, and PKR are also IFN inducible, elevated tonic IFN signaling in the TME may in fact represent a potential vulnerability exploitable not only by CBL0137, but also by future ADAR1 inhibitors (which will increase both A- and Z-form dsRNA-initiated immunostimulatory responses), and by ZBP1-activating viruses, such as the HSV-1 based oncolytic agent talimogene laherparepvec (T-vec) (Liu et al., Nat Med, 2019, 25, 95-102; Guo et al., Cell Death Dis, 2018, 9, 816; and Ribas et al., Cell, 2017, 170, 1109-1119.e1110).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety.

1. A method of treating cancer in a subject comprising administering to the subject in need thereof a compound, or pharmaceutically acceptable salt thereof, of any one of Formula I, and a checkpoint inhibitor, wherein:
the compound having Formula I comprises: 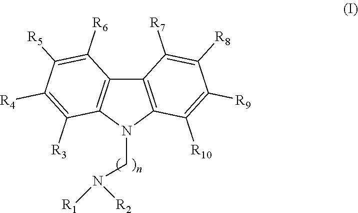
or a pharmaceutically acceptable salt thereof, wherein:
each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl;
each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and
n is 1, 2, or 3.

2. The method of claim 1, wherein the compound has Formula I: 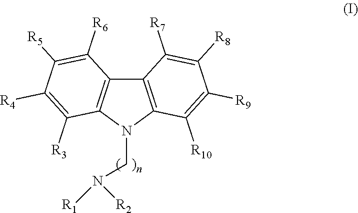
or a pharmaceutically acceptable salt thereof, wherein:
each of R₁ and R₂ is, independently, selected from the group consisting of hydrogen, substituted or unsubstituted alkyl, substituted or unsubstituted cycloalkyl, substituted or unsubstituted aryl, and substituted or unsubstituted sulfonyl;
each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted aryl, substituted or unsubstituted heteroaryl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and
n is 1, 2, or 3.

3. The method of claim 2, wherein each of R₁ and R₂ is, independently, selected from the group consisting of hydrogen, substituted or unsubstituted alkyl, and substituted or unsubstituted aryl.

4. The method of claim 2, wherein each of R₁ and R₂ is, independently, hydrogen or substituted or unsubstituted alkyl.

5. The method of claim 2, wherein each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio.

6. The method of claim 2, wherein each of R₃, R₄, R₅, R₆, R₇, R₈, R₉, and R₁₀ is, independently, selected from the group consisting of hydrogen, halogen, hydroxyl, nitro, substituted or unsubstituted alkyl, —C(═O)alkyl, substituted or unsubstituted alkenyl, substituted or unsubstituted alkoxy, and substituted or unsubstituted amine.

7. The method of claim 2, wherein each of R₃, R₄, R₅, R₆, R₇, R₈, R₉, and R₁₀ is, independently, selected from the group consisting of hydrogen, halogen, hydroxyl, substituted or unsubstituted alkyl, —C(═O)alkyl, and substituted or unsubstituted alkenyl.

8. The method of claim 2, wherein n is 2 or 3.

9. The method of claim 2, wherein:
each of R₁ and R₂ is, independently, selected from the group consisting of hydrogen, substituted or unsubstituted alkyl, and substituted or unsubstituted aryl;
each of R₃, R₄, R₅, R₆, R₇, R₈, R₉, and R₁₀ is, independently, selected from the group consisting of hydrogen, halogen, hydroxyl, cyano, nitro, substituted or unsubstituted alkyl, —C(═O)alkyl, substituted or unsubstituted alkenyl, substituted or unsubstituted alkoxy, substituted or unsubstituted amine, substituted or unsubstituted alkylamino, substituted or unsubstituted aminoalkoxy, and substituted or unsubstituted alkylthio; and
n is 1, 2, or 3.

10. The method of claim 2, wherein:
each of R₁ and R₂ is, independently, hydrogen or substituted or unsubstituted alkyl;
each of R₃, R₄, R₅, R₆, R₇, R₈, R₉, and R₁₀ is, independently, selected from the group consisting of hydrogen, halogen, hydroxyl, nitro, substituted or unsubstituted alkyl, —C(═O)alkyl, substituted or unsubstituted alkenyl, substituted or unsubstituted alkoxy, and substituted or unsubstituted amine; and
n is 2 or 3.

11. The method of claim 2, wherein:
each of R₁ and R₂ is, independently, hydrogen or substituted or unsubstituted alkyl;
each of R₃, R₄, R₅, R₆, R₇, R₈, R₉, and R₁₀ is, independently, selected from the group consisting of hydrogen, halogen, hydroxyl, substituted or unsubstituted alkyl, —C(═O)alkyl, and substituted or unsubstituted alkenyl; and
n is 2 or 3.

12. The method of claim 2, wherein:
each of R₁ and R₂ is, independently, hydrogen or substituted or unsubstituted alkyl;
each of R₃, R₄, R₅, R₆, R₇, R₈, R₉, and R₁₀ is, independently, selected from the group consisting of hydrogen, hydroxyl, substituted or unsubstituted alkyl, and —C(═O)alkyl; and
n is 2 or 3.

13. The method of claim 2, wherein the compound is:

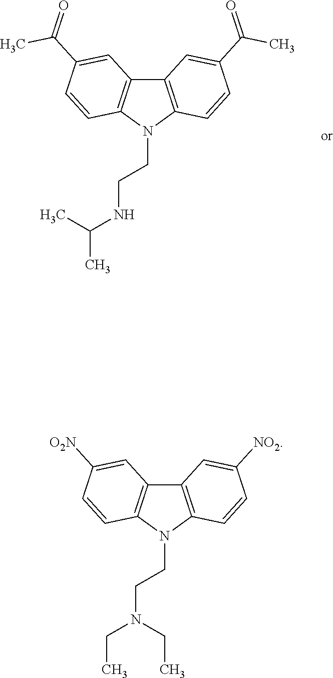 [SMILES: CC(=O)c1ccc2c(c1)c1cc(C(C)=O)ccc1n2CCNC(C)C ; CCN(CC)CCn1c2ccc([N+](=O)[O-])cc2c2cc([N+](=O)[O-])ccc21]

14.-37. (canceled)

38. The method of claim 1, wherein the checkpoint inhibitor is selected from the group consisting of an A2AR inhibitor, a B7-H3 inhibitor, a B7-H4 inhibitor, a BTLA inhibitor, a CTLA-4 inhibitor, an IDO inhibitor, a KIR inhibitor, a LAG3 inhibitor, a NOX2 inhibitor, a PD-1 inhibitor, a PD-L1 inhibitor, a PD-L2 inhibitor, a TIM-3 inhibitor, a VISTA inhibitor, and an SIGLEC7 inhibitor, or any combination thereof.

39. The method of claim 38, wherein the checkpoint inhibitor is selected from the group consisting of a BTLA inhibitor, a CTLA-4 inhibitor, an IDO inhibitor, a KIR inhibitor, a LAG3 inhibitor, a NOX2 inhibitor, a PD-1 inhibitor, a PD-L1 inhibitor, a PD-L2 inhibitor, a TIM-3 inhibitor, a VISTA inhibitor, and an SIGLEC7 inhibitor, or any combination thereof.

40. The method of claim 38, wherein the checkpoint inhibitor is selected from the group consisting of a CTLA-4 inhibitor, a LAG3 inhibitor, a NOX2 inhibitor, a PD-1 inhibitor, a PD-L1 inhibitor, a PD-L2 inhibitor, a TIM-3 inhibitor, a VISTA inhibitor, and an SIGLEC7 inhibitor, or any combination thereof.

41. The method of claim 38, wherein the checkpoint inhibitor is selected from the group consisting of a CTLA-4 inhibitor, a LAG3 inhibitor, a PD-1 inhibitor, a PD-L1 inhibitor, a PD-L2 inhibitor, and a VISTA inhibitor, or any combination thereof.

42. The method of claim 38, wherein the checkpoint inhibitor is selected from the group consisting of a CTLA-4 inhibitor, a LAG3 inhibitor, a PD-1 inhibitor, and a VISTA inhibitor, or any combination thereof.

43. The method of claim 38, wherein the checkpoint inhibitor is a PD-1 inhibitor selected from the group consisting of pembrolizumab, nivolumab, cemiplimab, JTX-4014, spartalizumab, camrelizumab, sintilimab, tislelizumab, toripalimab, dostarlimab, INCMGA00012, AMP-224, and AMP-514.

44. The method of claim 38, wherein the checkpoint inhibitor is a CTLA-4 inhibitor selected from the group consisting of ipilimumab and tremelimumab.

45. The method of claim 38, wherein the checkpoint inhibitor is a LAG3 inhibitor.

46. The method of claim 1, wherein the checkpoint inhibitor is a VISTA inhibitor.

47. The method of claim 1, wherein the cancer is selected from the group consisting of melanoma, bladder cancer, renal cancer, colon cancer, head and neck cancer, gastric cancer, lung cancer, and pancreatic cancer.

48. The method of claim 1, wherein the compound of Formula I is administered before the checkpoint inhibitor.

49. The method of claim 1, wherein the compound of Formula I is administered after the checkpoint inhibitor.

50. The method of claim 1, wherein the compound of Formula I is administered in the same composition as the checkpoint inhibitor.

51.-62. (canceled)
